(19) United States
(12) Patent Application Publication
(10) Pub. No.: US 2018/0327792 A1
(43) Pub. Date: Nov. 15, 2018

(54) Fermented Hydrolyzed Plant-Origin Material

(71) Applicant: The Quaker Oats Company, Chicago, IL (US)

(72) Inventors: Stephanie Marie BROWN, Chicago, IL (US); Sarah Marie CARVER, Schaumburg, IL (US); Yongsoo CHUNG, Lake in the Hills, IL (US); Juan GONZALEZ, Barrington, IL (US); Steven E. HAVLIK, Rolling Meadows, IL (US); Jeffrey D. MATHEWS, Naperville, IL (US); Valerie Christine SERSHON, Schaumburg, IL (US); Jin-E SHIN, Hoffman Estates, IL (US); Saravanan Suppiah SINGARAM, Barrington, IL (US)

(21) Appl. No.: 15/975,498

(22) Filed: May 9, 2018

Related U.S. Application Data
(60) Provisional application No. 62/504,449, filed on May 10, 2017.

(51) Int. Cl. C12P 7/56; A61K 35/741; A23L 7/104; A23L 2/38; A23P 10/40; A23P 10/25

(52) U.S. Cl. CPC C12P 7/56; A61K 35/741; A23L 7/104; A23V 2002/00; A23P 10/40; A23P 10/25; A23L 2/382

(57) ABSTRACT

A method and composition can provide fermented plant-origin material. The method can comprise several steps. A first step comprises hydrolyzing a plant-origin material to provide a hydrolyzed plant-origin material. A second step comprises providing a fermentation starter material comprising the hydrolyzed plant-origin material. A third step comprises fermenting the fermentation starter material to provide a fermented plant-origin material. Various compositions comprising a fermented plant-origin material are possible. In some embodiments, the fermented plant-origin material comprises a fermentation product produced by fermenting fermentation starter material, and the fermentation starter material comprises hydrolyzed plant-origin material. Even when the plant-origin material is hydrolyzed or hydrolyzed and fermented, certain desirable properties of the plant-origin material, for example, health benefits, nutrients, whole grain status, fiber content, or beta-glucan content, can be maintained. Additionally, the hydrolyzed or hydrolyzed and fermented plant-origin material can be provided with desirable organoleptic properties.

### CROSS-REFERENCE TO RELATED APPLICATIONS

This application is a nonprovisional application of, and claims priority to, U.S. Provisional Patent Application No. 62/504,449 filed on May 10, 2017, which is incorporated herein by reference in its entirety as an example.

### BACKGROUND

### Technical Field

The present invention relates to fermenting a composition comprising a hydrolyzed plant-origin material, for example, grain flour with hydrolyzed starch. As an example, the starch in a grain flour can be hydrolyzed, yet its soluble fiber content can be maintained, the concentration of the beta-glucan in the grain flour can be maintained and harm to the beta-glucan can be avoided. Moreover, the whole grain status of the grain can be maintained. As a result, in some embodiments, health benefits resulting from the plant-origin material, its soluble fiber concentration, beta-glucan concentration, whole grain status, fermented whole grain status or a combination thereof, can be maintained in a composition comprising the plant-origin material. Meanwhile, as a result of hydrolyzing and/or fermenting the plant-origin material, a composition comprising the fermented, hydrolyzed plant-origin material can also provide enhanced organoleptic properties, for example, reduced viscosity, reduced sliminess, desired taste, or a combination thereof. To illustrate potential uses of a composition as described herein, the composition can serve as a prebiotic, glycemic index reducer, immunity enhancer, energy enhancer, fiber source, soluble fiber source, nutrient additive, texture modifier, viscosity modifier, or a combination thereof.

### Background

Although existing products may be fermented or may comprise a plant-origin material with a hydrolyzed component, existing products tend to lack one or more potentially desirable features. For example, existing products can lack a desired concentration of grain, cereal grain, whole grain, legume, pulse, pomace, vegetable, fruit, soluble fiber, beta-glucan, associated health benefits, enhanced organoleptic properties, reduced viscosity, reduced sliminess, desired taste, fermentation metabolites, reduced pH, or a combination thereof.

### SUMMARY OF THE INVENTION

In one aspect, the invention provides a method comprising several steps. A first step comprises hydrolyzing a plant-origin material to provide a hydrolyzed plant-origin material. A second step comprises providing a fermentation starter material comprising the hydrolyzed plant-origin material. A third step comprises fermenting the fermentation starter material to provide a fermented plant-origin material.

In a second aspect, the invention comprises a composition formed by the method of the first aspect.

In a third aspect, the invention provides a composition comprising a fermented, hydrolyzed plant-origin material.

In a fourth aspect, the invention provides a composition comprising a fermented plant-origin material. The fermented plant-origin material comprises a fermentation product produced by fermenting fermentation starter material, and the fermentation starter material comprises hydrolyzed plant-origin material.

Other aspects, embodiments and features of the invention will become apparent from the following detailed description of the invention when considered in conjunction with the accompanying drawings. The accompanying figures are schematic and are not intended to be drawn to scale. In the figures, each identical or substantially similar component that is illustrated in various figures is represented by a single numeral or notation. For purposes of clarity, not every component is labeled in every figure. Nor is every component of each embodiment of the invention shown where illustration is not necessary to allow those of ordinary skill in the art to understand the invention.

### BRIEF DESCRIPTION OF THE DRAWINGS

The novel features believed characteristic of the invention are set forth in the appended claims. The invention itself, however, as well as a preferred mode of use, further objectives and advantages thereof, will be best understood by reference to the following detailed description of illustrative embodiments when read in conjunction with the accompanying drawings, wherein:

FIG. 1 is a schematic block flow diagram depicting an illustrative method for hydrolyzing a plant-origin material to provide a hydrolyzed plant origin material and fermenting the hydrolyzed plant-origin material to provide a fermented, hydrolyzed plant-origin material in accordance with one embodiment of an invention as described herein.

FIG. 2 is a schematic block flow diagram depicting an illustrative method comprising steps for adding an ingredient to a fermented, hydrolyzed plant-origin material to provide a food product and/or heat-treating a fermented, hydrolyzed plant-origin material or food product to provide a heat-treated product.

FIG. 3 is a schematic block flow diagram depicting an illustrative method comprising a step for adjusting a moisture concentration of a fermented, hydrolyzed plant-origin material to provide a moisture-adjusted fermented plant-origin material.

FIG. 4 is a schematic block flow diagram depicting an illustrative method comprising steps for dehydrating a fermented, hydrolyzed plant-origin material to provide a powder and/or adding a food product ingredient to a fermented, hydrolyzed plant-origin material or powder to provide a food product.

FIG. 5 is a schematic block flow diagram depicting an illustrative method comprising a step for packaging and/or refrigerating a fermented, hydrolyzed plant-origin material, a powder, or food product.

FIG. 6 is a schematic block flow diagram depicting an illustrative method comprising steps for hydrolyzing a composition, including extruding and deactivating the composition, to produce a hydrolyzed extrudate, pelletizing the hydrolyzed extrudate, drying the pellets to form dry pellets, and milling the dry pellets into flour.

FIG. 7 is a schematic illustration of an extruder that can be used in an exemplary method of the present disclosure.

FIG. 8 is a schematic block flow diagram depicting an illustrative method for combining water, plant origin material and an enzyme before hydrolyzing the plant-origin material to provide a hydrolyzed plant origin material and fermenting the hydrolyzed plant-origin material to provide a fermented, hydrolyzed plant-origin material in accordance with one embodiment of an invention as described herein.

### DETAILED DESCRIPTION

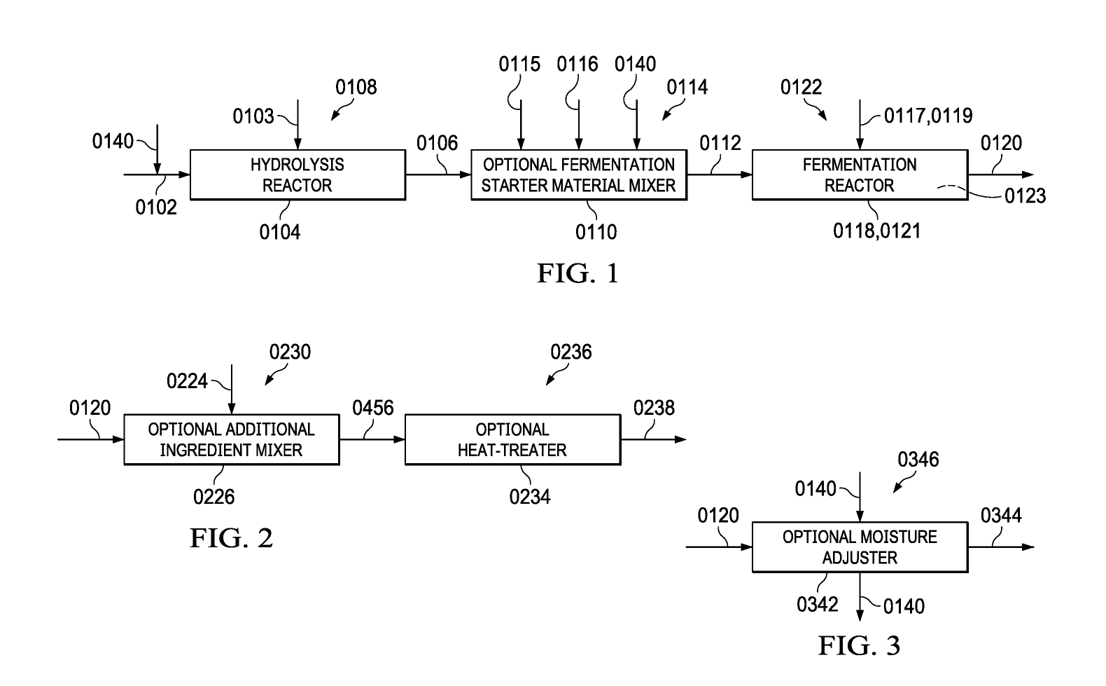

An embodiment of the invention will now be described with reference to FIG. 1. FIG. 1 is a schematic block flow diagram depicting an illustrative method in accordance with the invention described herein. The description of features with respect to FIG. 1 is also generally applicable to FIG. 8, although FIG. 8 illustrates an embodiment in which plant-origin material 0102, enzyme 0103 and optionally water 0140 are combined to form a hydrolysis starting material 0105 before the hydrolysis starting mixture is fed to a hydrolysis reactor 0104. The method illustrated in FIG. 1 comprises several steps. First, a hydrolyzing step comprises hydrolyzing 0108 a plant-origin material 0102 to provide a hydrolyzed plant-origin material 0106. This step can occur in a hydrolysis reactor 0104. Among other advantages, this step can be useful to increase the solubility or dispersibility of a solid product (e.g., powder or flour) produced in accordance with the present disclosure. This step can also be useful to reduce the viscosity of a flowable product (e.g., liquid product, slurry, semi-liquid product) produced in accordance with the present disclosure.

Second, a fermentation-starter-material providing step comprises providing 0114 a fermentation starter material 0112, which in turn comprises the hydrolyzed plant-origin material 0106. This step can occur in a fermentation starter material mixer 0110. Among other advantages, the fermentation-starter-material providing step can be useful to ensure that that hydrolyzed plant-origin material is in a form and condition that is conducive for fermentation. If the hydrolyzed plant-origin material is not in such a form or condition by itself, the temperature, pressure, and pH, of the hydrolyzed plant-origin material can be modified, additional ingredients may be added (e.g., water, nutrients, acid, base a combination thereof), and components (e.g., water, coarse solids, a combination thereof) may be removed or filtered out from the hydrolyzed plant-origin material to provide a fermentation-starter material that is conducive to fermentation.

Third, a fermentation step comprises fermenting 0122 the fermentation starter material 0112 to provide a fermented plant-origin material 0120. This step can occur in a fermentation reactor 0118. Among other advantages, this step can be useful to provide organoleptic properties associated with fermentation (e.g., taste, texture, smell, color) as well as health benefits associated with fermentation. The fermentation step can comprise forming a fermentation slurry, for example, by adding a fermenting agent 0117 to the fermentation material. In some embodiments, the fermented plant origin material 0120 can be provided with a total water mass concentration equal to about 70 to 95 wt. %, about 70 to 90 wt. %, about 80 to 90 wt. %, or about 83.5 to 86.5 wt. %.

With reference to FIG. 3, fourth, an optional moisture-adjustment step comprises adjusting 0346 a moisture concentration of the fermented plant-origin material 0120 to provide a moisture-adjusted fermented plant-origin material 0344, which can be a food product 0456, a powder, or a concentrate that can later be diluted to provide, for example, a beverage, at a desired strength for consumption. The moisture adjustment step can occur in a moisture adjuster 0342, for example, a mixer for adding water 0140 or a dryer or separator for removing water 0140. Although the moisture adjustment step can occur after the fermenting 0122, it can additionally or alternatively occur before, during or after an ingredient-adding step 0230, a heat-treating step 0236, a dehydrating step 0452, a food-product-ingredient-adding step 0460, or a combination thereof. Among other potential advantages, the moisture-adjustment step can be used to control the texture of the composition, to control the processability of the composition, to produce a beverage, a semi-liquid food, semi-solid food, spoonable food, or solid food, or to accomplish a combination thereof. In some embodiments, whether or not a moisture adjustment step is used, the fermented plant origin material can be provided with a total water mass concentration equal to about 70 to 95 wt. %, about 70 to 90 wt. %, about 80 to 90 wt. %, or about 83.5 to 86.5 wt. %.

With reference to FIG. 2, fifth, an optional ingredient-adding step comprises adding 0230 at least one ingredient 0224 to the fermented plant-origin material 0120 for example, to form a food product 0456. Examples of a food product include solid food, liquid food, semi-solid/semi-liquid food, spoonable product, food bar, yogurt, soup, beverage, etc. This ingredient-adding step can occur in an additional ingredient mixer 0226. Although the optional ingredient-adding step 0230 can occur after the fermenting step 0122, it can additionally or alternatively occur before, during or after a moisture adjustment step 0346, a heat-treating step 0236, a dehydrating step 0452, a food-product-ingredient-adding step 0460, or a combination thereof. Among other potential advantages, the ingredient-adding step can be useful to provide a product with desired organoleptic properties, processability, or health benefits.

Sixth, with reference to FIG. 2, an optional heat-treating step comprises heat-treating 0236, for example, pasteurizing, the fermented plant-origin material 0120 or the food product 0456 to provide a heat-treated product 0238, which can be a shelf-stable product. The heat-treating step 0236 can be accomplished by using a heat-treater 0234. Although the optional heat-treating step 0236 can occur after the ingredient-adding step 0230, it can additionally or alternatively occur before, during or after a moisture adjustment step 0346, an ingredient-adding step 0230, a dehydrating step 0452, a food-product-ingredient-adding step 0460, or a combination thereof. Among other potential advantages, the heat-treating step can be useful to sterilize a product (e.g., kill pathogens), to kill undesirable bacteria regardless of whether they are harmful, to pasteurize a product, or to provide the product in a shelf-stable form.

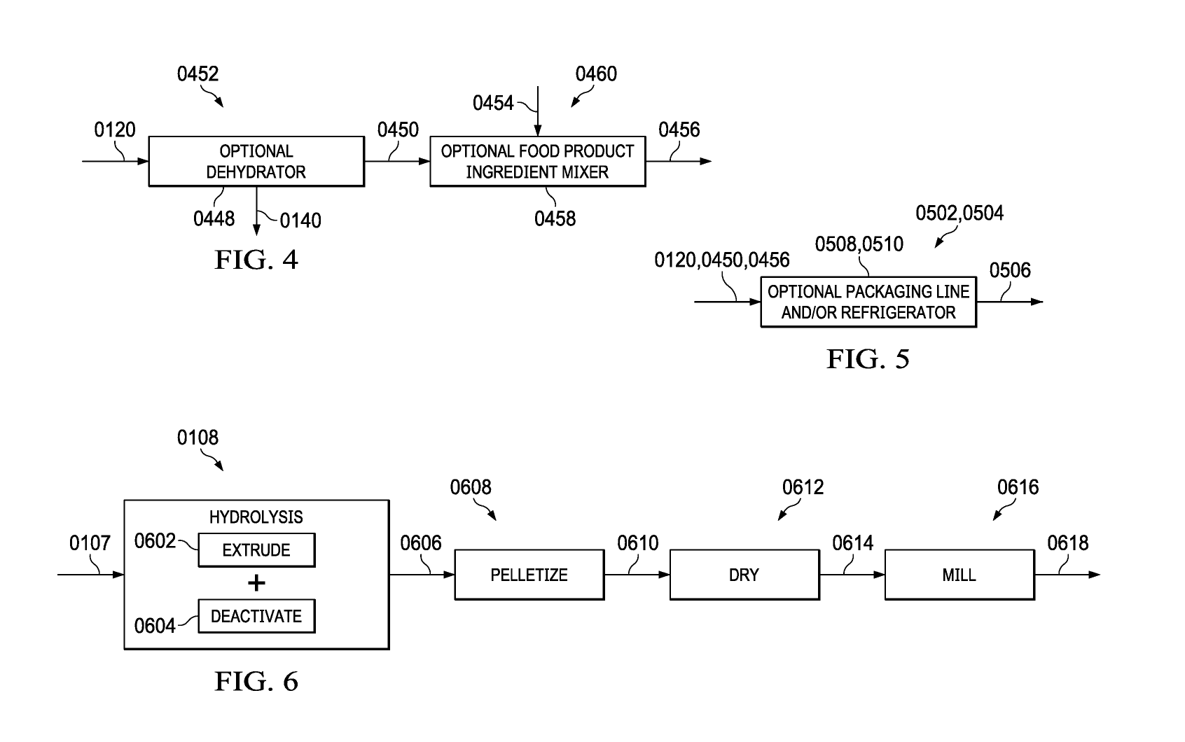

Seventh, with reference to FIG. 4, an optional dehydrating step comprises dehydrating 0452 the fermented plant-origin material 0120 to form a powder 0450. Examples of dehydrating include drying, vacuum-dehydrating, drying with heat, using a moisture separator, filtering, etc. The dehydrating step 0452 can occur in a dehydrator 0448 that removes water 0140 from the fermented plant-origin material 0120. Examples of dehydrators include a dryer, vacuum, separator (e.g., filter), and combinations thereof. Although the optional dehydrating step 0452 can occur after the fermenting step 0122, it can additionally or alternatively occur before, during or after a moisture adjustment step 0346, a heat-treating step 0236, a food-product-ingredient-adding step 0460, or a combination thereof. Among other potential advantages, the dehydrating step can be useful to provide the product in the form of a solid, a crisp or crunchy product, a powder, a flour, or a concentrate (e.g., that can later be diluted to provide, for example, a beverage, at a desired strength for consumption), or a combination thereof.

Eighth, with reference to FIG. 4, an optional food-product-ingredient-adding step comprises adding 0460 a powder 0450 to at least one food product ingredient 0454 to provide a food product 0456. Examples of a food product include solid food, liquid food, semi-solid/semi-liquid food, spoonable product, a food bar, yogurt, soup, a beverage, etc. Optionally, the powder 0450 comprises live culture and/or microorganisms (e.g., live microorganisms having probiotic properties). In some embodiments, the food-product-ingredient-adding step 0460 occurs in a food product ingredient mixer 0458. Although the optional food-product-ingredient-adding step 0460 can occur after the dehydrating step 0452, it can additionally or alternatively occur before, during or after a fermenting step 0122, a moisture adjustment step 0346, a heat-treating step 0236, a dehydrating step 0452, or a combination thereof. Among other potential advantages, the food-product-ingredient-adding step can be useful to provide a product with desired organoleptic properties, processability, or health benefits.

Ninth, with reference to FIG. 5, an optional packaging and/or refrigerating step comprises packaging 0502 and/or refrigerating 0504 the fermented plant-origin material 0120, powder 0450, or food product 0456 to provide a product 0506 with live microorganisms (e.g., live microorganisms having probiotic properties). The packaging 0502 and/or refrigerating step 0504 (e.g., freezing) can be accomplished using a packaging line 0508 and/or refrigerator 0510 (which can include a freezer). Although the optional packaging 0502 and/or refrigerating 0504 step can occur after the fermenting step 0122, it can additionally or alternatively occur before, during or after a moisture adjustment step 0346, a heat-treating step 0236, a dehydrating step 0452, a food-product-ingredient-adding step 0460, or a combination thereof. Among other potential advantages, packaging 0502 and/or refrigerating 0504 can be useful to facilitate transportation, to facilitate further processing, to avoid spoilage, or to maintain desirable organoleptic or health related properties.

With reference again to FIG. 1, the plant-origin material 0102 can be or comprise materials including, for example, a grain, a cereal grain, a legume, a pulse, a pomace, a vegetable, a fruit, a plurality of types of grains, a plurality of cereal grains, a plurality of legumes, a plurality of pulses, a plurality of pomaces, a plurality of vegetables, or a plurality of fruits. Moreover, the plant-origin material 0102 can also be or comprise any combination of these materials and/or any combination of portions of these materials, for example, solids (e.g., pulp), liquids (e.g., juice), or a combination thereof. In some embodiments, the plant-origin material 0102 is or comprises oat, a flour, a highly dispersible flour, or a combination thereof. In some embodiments, the plant-origin material 0102 comprises a protein concentrate. In some embodiments, the plant-origin material 0102 comprises a protein isolate.

With further reference to FIG. 1, the hydrolyzing step 0108 can comprise hydrolyzing 0108 starch, fiber, protein, or a combination thereof in the plant-origin material 0102. In some embodiments, the plant-origin material 0102 is in the form of an extruded pellet or a flour, which, for example, can be ground from an extruded pellet. Additionally, water 0140 can be added to the plant-origin material 0102 before the hydrolyzing 0108 the plant-origin material 0102. This can be useful, for example, if the plant-origin material 0102 is in the form of an extruded pellet or a flour.

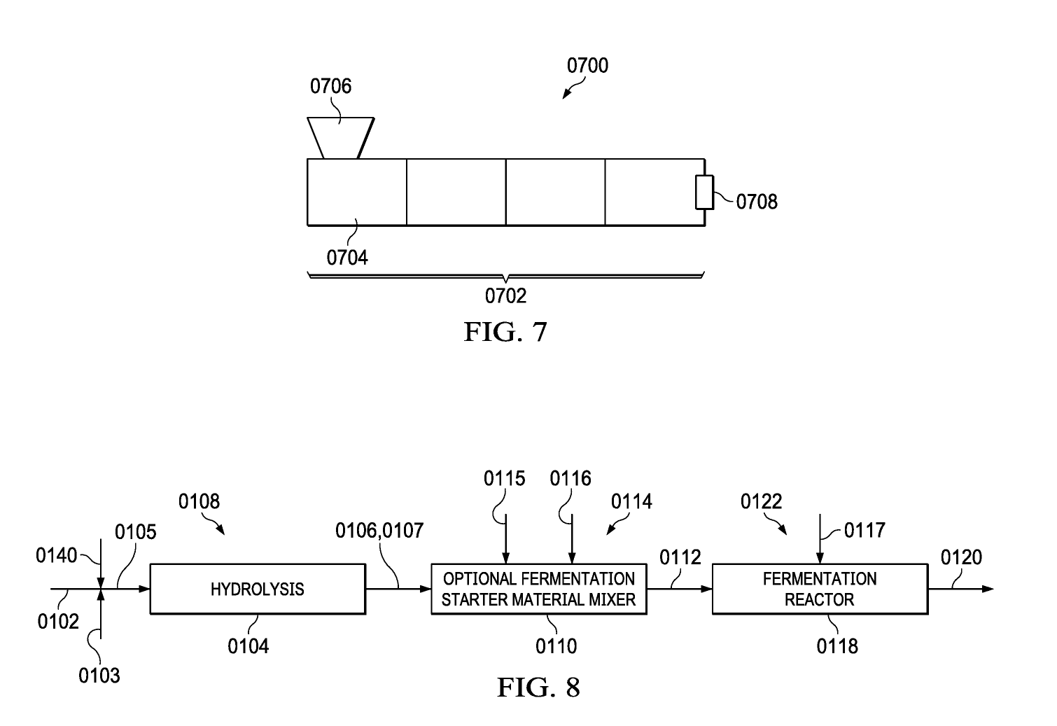

At least one enzyme 0103 (i.e., one enzyme or more enzymes) can be used to catalyze the hydrolysis of at least one macronutrient in the plant-origin material 0102. The at least one macronutrient can be starch, fiber, protein, or a combination thereof. Examples of fibers include soluble fiber, insoluble fiber or combinations thereof. Further examples of fiber include pectin, cellulose, and combinations thereof. The at least one enzyme 0103 can be selected from the group consisting of: alpha-amylase, pectinase, cellulase, and a combination thereof. With reference to FIG. 8, in some embodiments, the hydrolyzing 0108 comprises combining (e.g., mixing) the at least one enzyme 0103 with the plant-origin material 0102 and optionally water 0140 and to form a hydrolysis starting material 0105, which can be done before the hydrolysis starting material is fed to a hydrolysis reactor 0104 or an extruder 0700, which is schematically illustrated in FIG. 7. As illustrated in FIG. 1, the hydrolysis starting material can also be formed inside the hydrolysis reactor 0104 or an extruder 0700. The enzyme can be useful to catalyze hydrolysis of a macronutrient (e.g., starch) in the plant-origin material 0102. Consequently, the hydrolysis of the macronutrient (e.g., starch) in the hydrolysis starting material 0105 provides a hydrolyzed composition 0107 and the hydrolyzed composition 0107 comprises the hydrolyzed plant-origin material 0106. In some embodiments, the hydrolysis starting material 0105 comprises a total water mass concentration equal to about 25 to about 40 wt. %.

By way of example, the combining step can last for about 1 to about 5 minutes. In some embodiments, the combining step can last for about 3 to about 5 minutes. In some embodiments, the hydrolyzing 0108 comprises heating the hydrolysis starting material 0105 to a temperature equal to about 48 to about 100° C., or about 60 to about 83° C. to facilitate hydrolysis of the starch in the plant-origin material 0102. In some embodiments, the hydrolyzing 0108 lasts for a time that reduces the average molecular weight of starch in the plant-origin material 0102 to a hydrolyzed starch average molecular weight that is about 0.07 to about 95%, or 1 to 95%, or 6 to 95%, or 0.07 to 75%, 1 to 75%, or 6 to 75% of the average molecular weight of the starch in the plant-origin material 0102. In some embodiments, the hydrolyzing 0108 lasts for a time that reduces the peak molecular weight of starch in the plant-origin material 0102 to a hydrolyzed starch peak molecular weight that is about 6 to about 95% of the peak molecular weight of the starch in the plant-origin material 0102. For example, the peak molecular weight can be the highest molecular weight for starch detected in the plant origin material, the average molecular weight associated with the 1 wt. % of starch having the highest molecular weight, the lowest molecular weight of any starch in the 1 wt. % of the starch having the highest molecular weight, the number (or alternatively mass) average molecular weight of the starch in the one-hundred-thousand-Dalton range of molecular weights (e.g., 0 to 99,999 Dalton, 100,000 to 199,999, etc.) having the greatest number (or alternatively mass) of starch molecules that fall within the range, the number (or alternatively mass) average molecular weight of the starch in the ten-thousand-Dalton range of molecular weights (e.g., 0 to 9,999 Dalton, 10,000 to 19,999, etc.) having the greatest number (or alternatively mass) of starch molecules within the range, the number (or alternatively mass) average molecular weight of the starch in the one-thousand-Dalton range of molecular weights (e.g., 0 to 999 Dalton, 1,000 to 1,999, etc.) having the greatest number (or alternatively mass) of starch molecules within the range, or the molecular weight that is the statistical mode of the starch molecular weight distribution by number (or alternatively by mass). In some embodiments, the hydrolyzing 0108 lasts for about 0.5 to about 1.5 minutes or about 1 to about 1.5 minutes. Additionally, the hydrolyzed plant origin material can be plant-origin material 0102 comprising starch that has been hydrolyzed under controlled conditions to reduce the molecular weight of the starch while substantially avoiding hydrolysis of the starch to non-starch components to within a specified tolerance.

In some embodiments, the at least one enzyme 0103 is deactivated. By way of example, referring to FIG. 6, the hydrolyzing step 0108 can comprise deactivating 0604 the enzyme to provide the hydrolyzed plant-origin material 0106. In some embodiments, the deactivating step 0604 comprises heating the enzyme to a temperature sufficient to deactivate 0604 the enzyme, thereby providing the hydrolyzed plant-origin material 0106. For example, the deactivating 0604 can comprise heating the enzyme to about 100 to about 180° C., or about 100 to about 130° C., thereby providing the hydrolyzed plant-origin material 0106.

In some embodiments, the at least one enzyme 0103 can be deactivated so that no more than 5, 4, 3, 2, 1, 0.9, 0.8, 0.7, 0.6, 0.5, 0.4, 0.3, 0.2, 0.1 or 0.0 wt. % of the at least one macronutrient in the hydrolyzed plant-origin material 0106 has been converted to a component that no longer qualifies as the respective at least one macronutrient (e.g., starch or fiber can be converted to sugar and thus no longer qualify as starch or fiber, respectively). As an example, alpha-amylase can be used to hydrolyze starch, and the alpha-amylase can be deactivated so that no more than 5, 4, 3, 2, 1, 0.9, 0.8, 0.7, 0.6, 0.5, 0.4, 0.3, 0.2, 0.1 or 0.0 wt. % of the starch in the hydrolyzed plant-origin material 0106 has been converted to sugar.

With reference to FIG. 6, in some embodiments, the step of hydrolyzing 0108 the plant-origin material 0102, deactivating 0604 the enzyme, or a combination thereof comprises extruding 0602 the plant-origin material 0102, the enzyme and optionally water 0140. The extruding 0602 can take place in an extruder 0700 as depicted in FIG. 7. For example, the extruder 0700 can be a single- or twin-screw extruder 0700. The extruder can be fed through an inlet 0706 of the extruder. The extruder 0700 can comprise a barrel 0702 with at least one heated barrel section 0704. A wall of at least one heated barrel section 0704 comprises a wall temperature equal to about 60 to about 166, about 137 to about 166, about 137 to about 152 or about 137 to about 150° C. In some embodiments, the extruder 0700 comprises a barrel 0702 with a plurality of barrel sections 0704. Each of the plurality of barrel sections 0704 can comprise a wall temperature that differs from the wall temperature of the other barrel sections 0704 in the plurality of barrel sections 0704. In some embodiments, the hydrolyzed composition 0107 is extruded 0602 through a die assembly 0708 of the extruder 0700. In some embodiments, the hydrolyzed composition is provided to the die assembly 0708 at a die pressure equal to about 1700 to about 11700 kPa. Moreover, the die temperature can be about 60 to about 166, about 137 to about 166 or about 140 to about 166° C., to form a hydrolyzed extrudate 0606 as illustrated in FIG. 6. With reference to FIG. 6, the hydrolyzed extrudate, which is a form of hydrolyzed plant-origin material, can then be optionally pelletized 0608 to provide pellets 0610. The pellets can be optionally dried 0612 to provide dried pellets 0614. The hydrolyzed plant-origin material (e.g., hydrolyzed extrudate, pellets, or dried pellets) can be optionally milled 0616 to provide flour 0618. In some embodiments, the particle size of the flour can be measured using Malvern particle size analysis equipment (e.g., laser diffraction particle sizing equipment, for example, Malvern Mastersizer equipment). In some embodiments, the particle distribution of the particles in the flour are as follows. First, the smallest 10% of the particles by volume (Dv (10)), or alternatively by mass (Dm (10)) or number (Dn (10)) can have a size less than or equal to 10 microns+/−50, 30, 20, 10 or 5%. In other words Dx (10)=10 microns+/−50, 30, 20, 10 or 5%. Second, the smallest 50% of the particles by volume (Dv (50)), or alternatively by mass (Dm (50)) or number (Dn (50)), have a size less than or equal to 39 microns+/−50, 30, 20, 10 or 5%. In other words Dx (50)=39 microns+/−50, 30, 20, 10 or 5%. Third, in some embodiments, the smallest 90% of the particles by volume (Dv (90)), or alternatively by mass (Dm (90)) or number (Dn (90)), can have a size less than or equal to 124 microns+/−50, 30, 20, 10 or 5%. In other words Dx (90)=124 microns+/−50, 30, 20, 10 or 5%. In some embodiments, the volume (or alternatively mass) mean diameter of the particles (D[4,3]) in the flour can be equal to 59 microns+/−50, 30, 20, 10 or 5%. In some embodiments, about 90 to 100 wt. % of the particles in the flour 0618 can have a particle size less or equal to about 500, 450, 420, 400, 354, 300, 297, 210, 200, 105, 100, 90, 88, 53, 50, 46 or 44 microns and optionally greater than equal or equal to about 0.5, 1, 10, 20, 25, 30 or 32 microns. In some embodiments, about 90 to 100 wt. % of the particles in the flour 0618 can pass through a filter with a nominal size equal to about 500, 450, 420, 400, 354, 300, 297, 210, 200, 105, 100, 90, 88, 53, 50, 46 or 44 microns and optionally are retained by a filter with a nominal size equal to about 0.5, 1, 10, 20, 25, 30 or 32 microns. In some embodiments, 90 to 100 wt. % of the particles in the flour have a nominal US Mesh size less than or equal to 35, 40, 45, 50, 70, 140, 170, 270 or 325 and optionally have a nominal US Mesh size greater than or equal to 635, 500 or 450. In some embodiments, 90 to 100 wt. % of the particles in the flour pass through a screen having a nominal US Mesh size equal to 35, 40, 45, 50, 70, 140, 170, 270 or 325 and optionally are retained by a screen having a nominal US Mesh size equal to 635, 500 or 450.

In some embodiments, any beta-glucan in the fermented plant-origin material 0120 is structurally unchanged (or at least substantially structurally unchanged or no more than 10, 9, 8, 7, 6, 5, 4, 3, 2, or 1 wt. % of the beta-glucan is structurally changed) as a result of hydrolyzing 0108 the plant-origin material 0102, and/or the mass proportion of beta-glucan in the hydrolyzed plant-origin material 0106 is not reduced relative to a mass proportion of beta-glucan in the intact plant-origin material 0102 from which the hydrolyzed plant-origin material 0106 is derived, when the mass proportion of beta-glucan in the fermented plant-origin material 0120 is calculated excluding any materials that have been added to the plant-origin material 0102 to form the fermented plant-origin material.

In some embodiments, the mass ratio of starch:protein in the fermented plant-origin material 0120 is equal to: a mass ratio of starch:protein in the plant-origin material 0102 to within a tolerance of +/−30, 25, 20, 15, 10, 9, 8, 7, 6, 5, 4, 3, 2 or 1% of the mass ratio of starch:protein in the plant-origin material 0102; a mass ratio of starch:protein in the plant-origin material 0102 to within a tolerance of +/−30, 25, 20, 15, 10, 9, 8, 7, 6, 5, 4, 3, 2 or 1% of the mass ratio of starch:protein in the hydrolyzed plant-origin material 0106; or a combination thereof. In some embodiments, a mass ratio of fat:protein in the fermented plant-origin material 0120 is equal to: a mass ratio of fat:protein in the plant-origin material 0102 to within a tolerance of +/−30, 25, 20, 15, 10, 9, 8, 7, 6, 5, 4, 3, 2 or 1% of the mass ratio of fat:protein in the plant-origin material 0102; a mass ratio of fat:protein in the plant-origin material 0102 to within a tolerance of +/−30, 25, 20, 15, 10, 9, 8, 7, 6, 5, 4, 3, 2 or 1% of the mass ratio of fat:protein in the hydrolyzed plant-origin material 0106; or a combination thereof. In some embodiments, the mass ratio of beta-glucan:protein in the fermented plant-origin material 0120 is equal to: a mass ratio of beta-glucan:protein in the plant-origin material 0102 to within a tolerance of +/−30, 25, 20, 15, 10, 9, 8, 7, 6, 5, 4, 3, 2 or 1% of the mass ratio of beta-glucan:protein in the plant-origin material 0102; a mass ratio of beta-glucan:protein in the plant-origin material 0102 to within a tolerance of +/−30, 25, 20, 15, 10, 9, 8, 7, 6, 5, 4, 3, 2 or 1% of the mass ratio of beta-glucan:protein in the hydrolyzed plant-origin material 0106; or a combination thereof.

With reference to FIG. 1, providing 0114 a fermentation starter material 0112 can comprise hydrolyzing the plant-origin material 0102. Although, in some embodiments, providing 0114 a fermentation starter material 0112 comprises adding an additional component 0116 to the hydrolyzed plant-origin material 0106. The additional component 0116 can be additional carbohydrates, additional proteins, additional lipids, additional vitamins, additional minerals, and any combination thereof. Furthermore, as applicable, the additional components can be derived from plant, algae, or animal origin, for example, animal proteins, vegetable proteins, animal lipids, vegetable lipids, or a combination thereof. Moreover, the carbohydrates may be digestible, indigestible, soluble, insoluble, or a combination thereof. In some embodiments, fermentation starter material 0112 can comprise from about 5 to 25 wt. % or about 7 to 15 wt. % or about 10 to about 14 wt. % plant-origin material 0102; from about 0.5 to about 5 wt. % or about 1 to about 3 wt. % sugar (e.g., sucrose, dextrose, fructose, in the form of or derived from fruit pomace, in the form of or derived from vegetable pomace, or a combination thereof); and from about 76 to about 96 wt. % added water. In some embodiments, the fermentation starting material 0112 consists of plant-origin material 0102 (e.g., at a specified weight percentage), sugar (e.g., at a specified weight percentage), and water. In some embodiments, the fermenting 0122 occurs in a fermentation vessel 0121. For example, the fermentation starter material 0112 and fermentation culture 0119 can be mixed at a fermentation-starter-material to fermentation-culture mass ratio of about 5500:1 to about 4400:1, or optionally about 5000:1, to provide a fermentation slurry 0123. The fermentation slurry 0123 can be fermented to provide the fermented plant-origin material 0120. In some embodiments, the fermentation slurry 0123 comprises about 0.018 to about 0.022 wt. %, or optionally about 0.020 wt. %, fermentation culture 0119. For example, the fermentation slurry 0123 can comprise about 99.982 wt. % to about 99.978 wt. %, or optionally about 99.980 wt. %, fermentation starter material 0112. In some embodiments, the fermentation culture 0119 comprises lactobacillus cultures. In some embodiments, the fermenting 0122 comprises agitating the fermentation slurry 0123 in a fermentation vessel 0121. The agitating can be caused by rotating a shaft having at least one protrusion, rotating a shaft having at least one paddle, rotating an auger, rotating an impeller, or a combination thereof at about 100 to about 400 rpm or about 150 rpm in the fermentation slurry 0123. Optionally, the agitating can last for about 10 to about 21 hours or about 15 to about 21 hours. Optionally, the agitating can occur at about 35 to about 42° C. or about 40° C., or at about atmospheric pressure. In some embodiments, the agitating can be caused by an impeller with a relatively shorter pitch. The inventors realized that using an impeller with a shorter pitch helped to provide better mixing of the fermentation slurry for purposes of fermentation. For example, the shorter pitch enabled fermentation to proceed to a greater extent and to a lower pH, for example, a pH of 4.0, 3.9, 3.8 or lower, and optionally down to about 2.0, 2.1, 2.2, 2.3, 2.4 or 2.5. In some embodiments, the impeller can be a pitched blade, hydrofoil turbine, which, for example, the inventors realized worked better for mixing than a Rushton Turbine, which uses vertical paddles that are perpendicular to a direction of rotation. In some embodiments, the impeller can have a diameter of 114.3 mm+/−50, 40, 30, 20, or 10%.

Providing 0114 a fermentation starter material 0112 can also comprise adding an additional plant-origin material 0115 to the hydrolyzed plant-origin material 0106. The additional plant-origin material 0115 can be a grain, a cereal grain, a pulse, a legume, a pomace, a vegetable, a fruit, a plurality of types of grains, cereal grains, pulses, legumes, pomaces, vegetables, fruits, and a combination thereof.

In some embodiments, the additional plant-origin material 0115 is unhydrolyzed. Moreover, in some embodiments, the additional plant-origin material 0115 has not been subject to intentional hydrolysis, the additional plant-origin material 0115 has not been subject to significant hydrolysis, no more than 0.1, 0.2, 0.3, 0.4, 0.5, 1, 2, 3, 4, or 5 wt. % of at least one macronutrient (e.g., starch) in the additional plant-origin material 0115 has been hydrolyzed, the average molecular weight of the at least one macronutrient (e.g., starch) has decreased due to hydrolysis by no more than 0.1, 0.2, 0.3, 0.4, 0.5, 1, 2, 3, 4, or 5 wt. %, or a combination thereof. In some embodiments, avoiding hydrolysis of components or avoiding an undesirable degree of hydrolysis can be useful to maintain desired properties (e.g., organoleptic properties, health-related properties, whole-grain status, fermented whole grain status, or a combination thereof) or to enable a regulated health claim, or a combination thereof. As an example of fermented whole grain status, if a fermentation starter material comprises a plant-origin material with whole grain status, then fermenting the fermentation starter material results in a plant-origin material with fermented whole grain status. In some embodiments, the hydrolyzed plant-origin material, fermented plant-origin material, or both, can comprise a selection of or each component in an original set of components (e.g., principal nutrients, components comprising starch, fat, dietary fiber, protein, sugar, beta-glucan, etc.) at an original mass ratio relative to protein within a tolerance of +/−20%, 15%, 10%, 5%, 2% or 1%. For example, the original mass ratio can be the mass ratio of a selection of components or each component relative to protein at a time of harvesting, although it can also be at another reference time, for example, any time before hydrolyzing, fermenting or both. As further examples, the original mass ratio can correspond to a time before processes including separation of the anatomical components of the whole grain, grinding, cooking, gelatinization of the starch in the whole grain, hydrolysis of the starch in the whole grain, and/or any combination thereof.

In some embodiments of a hydrolyzed plant-origin material, the hydrolyzed plant-origin material comprises at least a portion of grain, and the at least a portion of grain is hydrolyzed-starch whole grain (e.g., oat, rice, wheat, sorghum, etc.) comprising gelatinized, hydrolyzed starch. Furthermore, the hydrolyzed-starch whole grain can have, within a tolerance of +/−20%, 15%, 10%, 5%, 2% or 1%, at least one mass ratio selected from the group consisting of: (i) a mass ratio of starch to protein equal to a mass ratio of starch to protein of unhydrolyzed whole grain equivalent in kind and condition to the hydrolyzed-starch whole grain; (ii) a mass ratio of fat to protein equal to a mass ratio of fat to protein of unhydrolyzed whole grain equivalent in kind and condition to the hydrolyzed-starch whole grain; (iii) a mass ratio of dietary fiber to protein equal to a mass ratio of dietary fiber to protein of unhydrolyzed whole grain equivalent in kind and condition to the hydrolyzed-starch whole grain; and (iv) any combination thereof. For example, in some embodiments, if alpha-amylase is used to catalyze the hydrolysis of starch, then the starch will by hydrolyzed, but not protein, fat or fiber.

Here, it is worthwhile to note that the microbial expression of protein, starch, cellulose, and fat degrading enzymes can be controlled by the design of a fermentation starter material, the matrix to be fermented, and/or the fermentation process. Accordingly, selectively degrading and/or hydrolyzing specific macronutrients (e.g., one or more macronutrients described herein) can be accomplished and/or avoided as desired. Additionally, in some embodiments, one or more macronutrients can be utilized for cell survival (e.g., of microorganisms) and/or to produce desired metabolites (e.g., as short chain organic acids and aldehydes), for example, as a result of cell metabolism and/or fermentation. These processes can be tailored to provide a fermented plant-origin material with one or more desired properties (e.g., a desired property described herein or combination thereof). For example, the fermented plant-origin material can comprise a fermentation metabolite, for example, lactic acid, exo-polysaccharides that can be indigestible and have prebiotic properties, volatile components (e.g., acetaldehyde, acetone, 2-butanone, diacetyl, 2,3-pentanedione, acetoin, 1-hexanol, acetic acid, butanoic acid, hexanoic acid, dimethyl sulfide, ethanol or a combination thereof), or a combination thereof.

In some embodiments, the additional plant-origin material 0115 is hydrolyzed. For example, the additional plant-origin material 0115 has been subject to intentional hydrolysis, the additional plant-origin material has been subject to significant hydrolysis, at least 0.1, 0.2, 0.3, 0.4, 0.5, 1, 2, 3, 4, 5, 10, 20, 30, 40, 50, 60, 70, 80, 90, 95, 96, 97, 98, 99 or 100 wt. % of at least one macronutrient (e.g., starch) in the additional plant-origin material 0115 has been hydrolyzed, the average molecular weight of the at least one macronutrient (e.g., starch) has decreased due to hydrolysis by at least 0.1, 0.2, 0.3, 0.4, 0.5, 1, 2, 3, 4, 5, 10, 20, 30, 40, 50, 60, 70, 80, 90, 95, 96, 97, 98, 99 wt. %, or a combination thereof. In some embodiments, purposely causing hydrolysis of components, for example full hydrolysis or at least a desirable degree of hydrolysis, can be useful to maintain provide properties (e.g., health-related properties, organoleptic properties, viscosity, enhanced processability or a combination thereof) while simultaneously preserving or maintaining other desirable properties (e.g., organoleptic properties, health-related properties, whole-grain status, fermented whole grain status, properties required to make a regulated health claim, or a combination thereof).Moreover, in some embodiments, providing 0114 a fermentation starter material 0112 or fermenting 0122 the fermentation starter material comprises adding a fermenting agent 0117 to the fermentation starter material 0112 (e.g., thereby providing a fermentation slurry 0123 comprising the fermentation starter material and the fermenting agent) to cause the fermenting 0122 of the fermentation starter material 0112 (e.g., in the fermentation slurry 0123). Of course, as a skilled person would understand upon reading this disclosure, the fermenting 0122 of the fermentation starter material can effectively begin at the same time or after the fermenting agent 0117 is added to the fermentation starter material, depending upon whether the conditions of the combined fermentation starter material and fermenting agent (e.g., in a fermentation slurry) are conducive to fermentation. As examples, the fermenting agent 0117 can be yeast, bacteria, or a combination thereof. Examples of yeast include Saccharomyces, Candida, Kluyveromyces, and a combination thereof. Examples of bacteria include Lactobacillus species, for example, Lactobacillus acidophilus, Lactobacillus delbruckii subsp. bulgaricus, Lactobacillus paracasei, Lactobacillus plantarum, Lactobacillus sanfrancisco, other lactic acid bacteria, for example, Streptococcus thermophilus, Bifidobacterium, Lactococcus species, Leuconostocs, Pediococcus, or any combination thereof. In some embodiments, the bacteria is a bacteria that is used for lactic acid fermentation. In some embodiments, the bacteria is a bacteria that has beta-glucanase activity of less than a desired amount. In other embodiments the bacteria or microorganisms can have beta-glucanase activity as long as the beta-glucanase activity is not expressed during fermentation. For example, it can be desirable to have low enough beta-glucanase activity (in general or expressed under fermentation conditions) to maintain a characteristic from the plant-origin material or fermentation starter material or fermentation slurry to the fermented plant-origin starter material (e.g., fermented plant-origin material or fermented fermentation starter material), which can include whole grain status, fermented whole grain status, a desired beta-glucan content, a desired soluble beta-glucan content, a desired soluble fiber content, some other status or entitlement to a health claim (e.g., as described herein). In some embodiments, it can be desirable to avoid culture, microorganism, compound, enzyme, hydrolysis process, fermentation process, or combination thereof that selectively hydrolyzes beta-glucan, has beta-glucanase activity, expresses beta-glucanase activity during fermentation, or a combination thereof. In some embodiments, microorganisms (e.g., bacteria) are selected so that during fermentation, the selected bacteria express limited beta-glucan activity during fermentation so that the level of beta glucan in the composition after fermentation is at least (and/or no more than) 30, 40, 50, 60, 70, 80, 90, 95, 96, 97, 98, or 99 wt. % the beta-glucan present in the fermentation starter material that is fermented to provide the composition.

With reference to FIG. 1, the fermenting step 0122 can occur under specified fermentation conditions. For example, the fermenting can occur at a pressure of 100-500, or 100-400, or 100-300, or 100-200, or 100-150 kPa (e.g. 101.325 kPa); at a temperature of 25-45, 25-40, 25-35, 25-30, 30-35, 35-40, 40-45, or 35-45° C.; under stirring, mixing, or agitation; at a pH of 5.5-7.8 at the start of fermentation; at a desired redox potential; at a desired ionic strength; after or at the time of inoculating the fermentation starter material to provide an inoculated fermentation starter material comprising 10̂5-10̂8 colony forming units per milliliter (CFU/ml) of the inoculated fermentation starter material; for 1-36, 1-30, 1-25, 1-20, 1-15, 1-10, 1-5 hours or more than 36 hours; or a combination thereof.

In some embodiments, the fermenting step 0122 comprises a yeast fermentation step. For example, the yeast fermentation step can comprise adding yeast to the fermentation starter material 0112 (e.g., to provide a fermentation slurry comprising the fermentation starter material and the yeast). Among other things, this can provide the fermented plant-origin material 0120 with yeast-fermentation flavors.

In some embodiments, the fermenting step 0122 comprises a bacterial fermentation step. For example, the bacterial fermentation step can comprise adding bacteria (e.g., by adding culture comprising one or more bacteria strains) to the fermentation starter material 0112 (e.g., to provide a fermentation slurry comprising the fermentation starter material and the bacteria). Among other things, this can provide the fermented plant-origin material 0120 with bacterial-fermentation flavors.

In some embodiments, the fermenting step 0122 comprises a yeast fermentation step followed by a bacterial fermentation step.

In some embodiments, beta-glucan in the fermented plant-origin material 0120 is structurally unchanged relative to the structure of beta-glucan in the plant-origin material 0102 and/or relative to the structure of beta-glucan in the hydrolyzed plant-origin material 0106.

In some embodiments, a mass proportion of beta-glucan in the fermented plant-origin material 0120 is not reduced relative to a mass proportion of beta-glucan in the intact plant-origin material 0102 from which the hydrolyzed plant-origin material 0106 is derived when the mass proportion of beta-glucan in the fermented plant-origin material 0120 is calculated excluding any materials that have been added to the plant-origin material 0102 to provide the fermented plant-origin material 0120.

With reference to FIG. 2, in some embodiments, the optional ingredient-adding step 0230 can comprise adding at least one ingredient 0224 to the fermented plant-origin material 0120. The at least one ingredient can be an additional liquid selected from the group consisting of water, milk, a dairy milk, a non-dairy milk, a vegetable juice, a fruit juice, and a combination thereof. Additionally or alternatively, at least one ingredient 0224 can be selected from the group consisting of a sweetener, sugar, sucrose, natural sweeteners, low calorie sweeteners, no calorie sweeteners, flavors (e.g, vanilla), a protein (e.g., plant protein or dairy protein), and a combination thereof.

Upon reading the present disclosure, a person skilled in the art will understand that the methods described herein can be used to produce a variety of products or compositions and these products or compositions are also encompassed within the scope of the present application.

Accordingly, in some embodiments the present invention provides a composition comprising a fermented, hydrolyzed plant-origin material 0120. For example, the fermented, hydrolyzed plant-origin material 0120 can be provided by fermenting a hydrolyzed plant-origin material 0106. In turn, the hydrolyzed plant origin material 0106 can be provided by hydrolyzing a plant-origin material 0102. As examples, the plant-origin material 0102 described herein can be a grain, a cereal grain, a legume, a pulse, a pomace, a vegetable, a fruit, a plurality thereof, or a combination thereof. Optionally, after hydrolysis, the hydrolyzed plant-origin material 0106 comprises at least one hydrolyzed macronutrient, which can be hydrolyzed starch, hydrolyzed fiber, hydrolyzed protein, or a combination thereof.

In some embodiments, the invention provides a composition comprising fermented plant-origin material 0120. The fermented plant-origin material 0120 comprises a fermentation product produced by fermenting 0122 fermentation starter material 0112 (e.g., in a fermentation slurry comprising the fermentation starter material), and the fermentation starter material 0112 comprises hydrolyzed plant-origin material 0106. Optionally, the hydrolyzed plant-origin material 0106 comprises a hydrolysis product produced by hydrolyzing 0108 a plant-origin material 0102.

The hydrolyzed plant-origin material useful in compositions and process of the present disclosure can take various forms. For example, the hydrolyzed plant-origin material 0106 can comprise a hydrolysis product produced by hydrolyzing 0108 at least one macronutrient in a plant-origin material 0102. In some embodiments, the at least one macronutrient can be starch, fiber, protein, or a combination thereof. Accordingly, the hydrolysis product can comprise at least one hydrolyzed macronutrient selected from the group consisting of hydrolyzed starch, hydrolyzed fiber, hydrolyzed protein, and a combination thereof.

In some embodiments, the hydrolyzed plant-origin material 0106 has been subject to intentional hydrolysis or significant hydrolysis. Additionally, in some embodiments, at least 0.1, 0.2, 0.3, 0.4, 0.5, 1, 2, 3, 4, 5, 10, 20, 30, 40, 50, 60, 70, 80, 90, 95, 96, 97, 98, 99 or 100 wt. % of each of at least one macronutrient (e.g., starch) in the hydrolyzed plant-origin material 0106 has been hydrolyzed.

In some embodiments, the average molecular weight of each of the at least one macronutrient (e.g., the starch) in the hydrolyzed plant origin material has decreased due to hydrolysis by at least 0.1, 0.2, 0.3, 0.4, 0.5, 1, 2, 3, 4, 5, 10, 20, 30, 40, 50, 60, 70, 80, 90, 95, 96, 97, 98, 99 wt. %, or a combination thereof. Among other advantages, this can be useful to provide a liquid or semi-liquid composition comprising the hydrolyzed plant-origin material with a relatively reduced viscosity. Moreover, the inventors have discovered that the reduced viscosity of hydrolyzed whole grain oats combined with fermentation starter material enables enhanced fermentation of a material comprising whole grain oats, a lower pH (e.g., 4.0, 3.9, 3.8 or less, and optionally down to about 2.0, 2.1, 2.2, 2.3, 2.4 or 2.5) for the resulting fermented plant-origin material, a higher concentration of whole grain in the fermented product, or a combination thereof. As an example of a hydrolyzed plant-origin material with a controlled and/or lower viscosity, the hydrolyzed plant-origin material 0106 can comprise a Rapid Visco Analyzer (“RVA”) peak viscosity equal to no more than 2500 or 2000 cP and optionally at least 1, 5, 10, 15, 20, 25, 30, 40, 50, 60, 70, 80, 90, 100, 200, 300, 400, 500, 600, 700, 800, 900, 1000, or 1500 cP. The inventors realized that viscosities below 2500 or 2000 cP are easier to process to achieve a desired degree of fermentation. Moreover, the fermented plant-origin material 0120 can comprise a viscosity at 25° C. equal to no more than 7500 or 7000 cP and optionally at least 2,000, 2,500, 4,500 or 5,000 cP. For example, the fermented plant-origin material 0120 can comprise a viscosity at 25° C. equal to no more than 2500 cP. In some embodiments, the viscosity is measured using rheological tests, for example, temperature sweeps. The viscosity can decrease with increasing temperature.

Rapid Visco Analyzer (“RVA”) peak viscosity of a composition (e.g., hydrolyzed plant-origin material) can be measured using the following protocol. First, a mixture is formed consisting of the composition and a remainder of water. Water is added in an amount to provide the mixture with 14.3 wt. % solids. In other words, if the mixture were completely dehydrated by evaporating away the moisture, 14.3 wt. % solids would remain.

Second, the mixture is mixed by turning a shaft with a paddle at 500 rpm for 5 seconds (e.g., so the composition is fully dispersed in the water to form a dispersion and generally homogeneous mixture, and to avoid clumps that can cause viscosity measurement errors).

Third, the dispersion is continuously mixed by turning a shaft with a paddle at 160 rpm and the viscosity of the dispersion is continuously measured while subjecting the dispersion to the following temperature profile: (i) holding the dispersion at about 25° C. for about 2 min; (ii) heating the dispersion to about 95° C. over about 5 minutes; (iii) holding the dispersion at about 95° C. for about 3 minutes; (iv) cooling the dispersion from about 95° C. to about 25° C. over about 5 minutes; (v) holding the dispersion at about 25° C. for about 3 min. The RVA peak viscosity is the maximum viscosity measured during steps (ii) and (iii).

Using a method such as the RVA peak viscosity measurement protocol can be useful, for example, to provide a way to compare the viscosity of compositions that are consumed after their starch has been gelatinized. This is so because the RVA peak viscosity measurement protocol involves heating and hydrating the composition, which gelatinizes starch in the composition if the starch has not already been gelatinized.

As explained herein, a hydrolyzed plant-origin product can be fermented. Accordingly, it can be desirable to form a fermentation starter material that comprises the hydrolyzed plant-origin material 0106. In some embodiments, the fermentation starter material 0112 further comprises an additional plant-origin material 0115, for example, a grain, a cereal grain, a pulse, a legume, a pomace, a vegetable, a fruit, a plurality thereof, or a combination thereof. As an example, the additional plant-origin material can comprise a plurality of types of grains, for example, oat and barley. As another example, the additional plant-origin material can comprise a plurality of types of grains, a pulse, and a plurality of types of vegetables. For example, it can be useful to combine types of grains and/or pulses to provide a more complete source of protein.

In some embodiments, the composition comprises at least one enzyme 0103 (e.g., deactivated enzyme), for example, alpha-amylase, pectinase, cellulase or a combination thereof. This enzyme can be active or deactivated. For example, in some embodiments, the fermentation starter material 0112 comprises at least one deactivated enzyme selected from the group consisting of: deactivated alpha-amylase, deactivated pectinase, deactivated cellulase and a combination thereof. It can be advantageous for the enzyme to be deactivated to stop the catalysis of a reaction, for example, a hydrolysis reaction, which can, in turn, help control the degree of hydrolysis in a composition. In some embodiments, beta-amylase and alpha-amylase can be used to catalyze hydrolysis of starch and to provide the hydrolyzed plant-origin material such that the hydrolyzed plant-origin material is not whole grain.

In some embodiments, the composition comprises fermentation-derived molecules selected from the group consisting of: organic acids (e.g., lactic acid), esters, alcohols, aldehydes, ketones, antimicrobial molecules, epoxypolysaccharides, and a combination thereof. In some embodiments, the selection of cultures, the formulation design for the fermentation media, the fermentation conditions or a combination thereof provide definition to the array of molecules that are present in the final fermented material.

In some embodiments of a composition, the plant-origin material 0102 and/or the hydrolyzed plant-origin material 0106 is grain or whole grain. For example, the hydrolyzed plant-origin material 0106 can be derived from intact grain caryopses. In the case of a grain-based plant-origin material, it can be advantageous to hydrolyze the starch to reduce the viscosity of any liquid or semi-liquid including the grain-based plant-origin material. Accordingly, in some embodiments, the average molecular weight of the hydrolyzed starch can be reduced by at least 30%, 40%, 50%, 60% or 65% relative to the average molecular weight of the starch in the intact grain caryopses.

In some embodiments, it can be advantageous to avoid overly processing a grain-based composition and thus causing the grain-based composition to lose certain desirable properties (e.g., organoleptic and/or health-related properties). For this or other reasons, it can be useful to provide a basis for determining whether a grain-product that has undergone some processing is still sufficiently similar to an original whole grain product to maintain certain desirable properties, for example, whole grain status or fermented whole grain status.

With this in mind, it is useful to note that intact grain caryopses comprise principal anatomical components, namely a starchy endosperm, a germ and a bran. Moreover, these principal anatomical components are present in the intact grain caryopses in a first set of relative component proportions. As an example, the first set of relative component proportions can comprise (i) the mass of starchy endosperm divided by the mass of germ, (ii) the mass of starchy endosperm divided by the mass of bran, (iii) the mass of bran divided by the mass of germ, (iv) the mass of any one principal anatomical component divided by the mass of any other principal anatomical component, or (v) a combination thereof.

Having established a first set of relative component proportions as a reference point, similar proportions for a somewhat processed product can be calculated and the result provides a useful framework for comparison. For example, the principal anatomical components described above can be present in a second set of relative component proportions in a hydrolyzed plant-origin material 0106. In some embodiments, each proportion in the second set of relative component proportions in the hydrolyzed plant-origin material 0106 is equal to the corresponding proportion in the first set of relative component proportions in the intact grain caryopses to within a specified tolerance.

For example, in some embodiments, the principal anatomical components are present in the same, approximately the same, or +/−5, 4, 3, 2, 1, 0.9, 0.8, 0.7, 0.6, 0.5, 0.4, 0.3, 0.2, 0.1 or 0.0% of the relative proportions as they exist in the intact caryopses from which the hydrolyzed plant-origin material 0106 is derived.

To provide an illustration, the mass of starchy endosperm divided by the mass of germ in the intact grain caryopses can be a value X and the mass of starchy endosperm divided by the mass of germ in the hydrolyzed plant-origin material can be the value X+/−5%, where X+/−5% indicates a range from (X minus 5% of X) to (X plus 5% of X). In some embodiments, as long as the proportion in the second set of relative component proportions is equal or sufficiently close to the corresponding proportion in the first set of relative component proportions, then certain desirable properties can be effectively maintained.

In addition to or as an alternative to using the principal anatomical components of intact caryopses, the principal nutrients of intact caryopses can also be used as a frame of reference for determining the presence of desirable properties in a composition. For example, in some embodiments, the hydrolyzed plant-origin material 0106 in a composition is derived from intact plant-origin material 0102 (e.g., grain caryopses), and the intact plant-origin material (e.g., grain caryopses) comprises principal nutrients, for example, starch, fat, protein, dietary fiber, beta-glucan, and sugar. Moreover, the principal nutrients can be present in a first set of relative nutrient proportions in the intact plant-origin material (e.g., grain caryopses). For example, the first set of relative nutrient proportions can comprise (i) the mass of starch divided by the mass of fat, (ii) the mass of starch divided by the mass of protein, (iii) the mass of starch divided by the mass of dietary fiber, (iv) the mass of starch divided by the mass of beta-glucan, (v) the mass of starch divided by the mass of sugar, (vi) the mass of any one principal nutrient divided by the mass of another principal nutrient, or (vii) a combination thereof.

Additionally, in a hydrolyzed plant-origin material 0106, the principal nutrients can be present in a second set of relative nutrient proportions. In some embodiments, each proportion in the second set of relative nutrient proportions is equal or approximately equal to the corresponding proportion in the first set of relative proportions. Moreover, in some embodiments, each proportion in the second set of relative nutrient proportions is equal to the corresponding proportion in the first set of relative proportions +/−5, 4, 3, 2, 1, 0.9, 0.8, 0.7, 0.6, 0.5, 0.4, 0.3, 0.2, 0.1 or 0.0%. For example, the mass of starch divided by the mass of fat in intact grain caryopses can be a value Y and the mass of starch divided by the mass of fat in the hydrolyzed plant-origin material can be a value Y+/−5%, where Y+/−5% indicates a range from (Y minus 5% of Y) to (Y plus 5% of Y).

In some embodiments, it is advantageous to provide a desired amount of beta-glucan in a hydrolyzed product. For example, in some embodiments, the hydrolyzed plant-origin material 0106 comprises 3 to 5, or 3.7 to 4 wt. % beta-glucan. It can also be desirable to avoid undesirable changes in the structure of beta-glucan as a result of hydrolysis. Accordingly, in some embodiments, the hydrolyzed plant-origin material 0106 is derived from intact grain caryopses comprising beta-glucan; and the beta-glucan in the hydrolyzed plant origin material or fermented plant-origin material is structurally unchanged relative to the beta-glucan in the intact caryopses. Additionally, in some embodiments, the hydrolyzed plant-origin material 0106 is derived from intact grain caryopses comprising beta-glucan; and at least (and/or no more than) 30, 40, 50, 60, 70, 80, 90, 95, 96, 97, 98, or 99% the beta-glucan in the hydrolyzed plant origin material or fermented plant-origin material is structurally unchanged relative to the beta-glucan in the intact caryopses.

Much as it can be useful to avoid hydrolyzing components at all or beyond a certain degree, it can also be useful to ensure that some components are hydrolyzed to a desired degree. For example, in some embodiments of a composition as described herein, at least 50, 60, 70, 80, 90, 95, 96, 97, 98, 99 or 100 wt. % of at least one macronutrient (e.g., starch) in the hydrolyzed plant-origin material 0106 is hydrolyzed (e.g., in the form of hydrolyzed starch). In some embodiments of a hydrolyzed plant-origin material, for example, where the at least one hydrolyzed macronutrient is hydrolyzed starch, no more than (and/or at least) 5, 4, 3, 2, 1, 0.9, 0.8, 0.7, 0.6, 0.5, 0.4, 0.3, 0.2, 0.1 or 0.0 wt. % of starch in hydrolyzed plant-origin material 0106 has been converted to sugar. Moreover, in some embodiments, the average molecular weight of hydrolyzed starch in the hydrolyzed plant-origin material 0106 is 1.7-2.0×106 Dalton. In some embodiments, the average molecular weight of the hydrolyzed starch molecules can be reduced to a fraction of the original average molecular weight (e.g., no more than about 60%, 50%, 40%, 30%, 20%, 10%, 9%, 8%, 7%, 6%, 5%, 4%, 3%, 2%, or 1% of the original molecular weight). This is so, because, for example, the starch molecules can be selectively reduced (e.g., using enzymes with only endo activity) in molecular weight to the smallest molecules that still constitute starch, but without being converted into molecules that are not starch, for example, sugar (e.g., monosaccharides or disaccharides).

In some embodiments, the average molecular weight of the gelatinized, hydrolyzed starch molecules in the composition is a fraction of the molecular weight of gelatinized, unhydrolyzed starch molecules equivalent (e.g., in kind and condition) to the gelatinized, hydrolyzed starch molecules, except that the gelatinized, unhydrolyzed starch molecules have not been hydrolyzed. For example, the fraction can be selected from the group consisting of about 0.90 to 0.47, 0.80 to 0.47, 0.70 to 0.47, 0.60 to 0.47, 0.50 to 0.47, less than about 0.90, less than about 0.80, less than about 0.70, less than about 0.60, less than about 0.50, and any range formed from values contained in the listed ranges.

In some embodiments, the composition comprises a mass concentration of fermented plant-origin material 0120, a mass concentration of hydrolyzed plant-origin material, or a mass concentration of both equal to 1-100%, 5-95%, 10-90%, 20-80%, 30-70%, 40-60%, 1-5%, 5-10%, 10-20%, 20-30%, 30-40%, 40-50%, 50-60%, 60-70%, 70-80%, 80-90%, 90-95%, 95-100%, or a combination thereof.

As can be seen, the plant-origin material that is hydrolyzed and fermented in the present disclosure can vary and provide a wide range of desirable properties for a product composition. Additionally, a product composition can comprise an additional plant-origin material 0115 selected from the group consisting of a grain, a cereal grain, a pulse, a legume, a pomace, a vegetable, a fruit, a plurality thereof, and a combination thereof.

Moreover, the composition can comprise an additional ingredient selected from the group consisting of: additional carbohydrates, additional proteins, additional lipids, additional vitamins, additional minerals, and a combination thereof. The additional ingredient or ingredients can be useful, for example, to provide the composition with additional desirable properties (e.g., organoleptic properties or health-related properties).

Further examples of, methods for making, systems for making, or apparatuses for making hydrolyzed, plant-origin material, additional plant-origin material, a grain, a cereal grain, a legume, a pulse, a pomace, a vegetable, a fruit, a plurality thereof, or a combination thereof will now be described with reference to several documents, all of which are incorporated herein by reference in their entirety as examples. As a first example, U.S. patent application Ser. No. 12/056,598, entitled “Hydrolyzed, Spray Dried, Agglomerated Grain Powder and Drinkable Food Products,” was published as U.S. Patent Application Publication No. 2008/0260909 A1 and issued as U.S. Pat. No. 8,241,696, which are all hereby incorporated by reference in their entirety as examples. In one aspect, U.S. Pat. No. 8,241,696 includes or can be modified to include a drinkable food product comprising water and about 5 wt % to about 15 wt % hydrolyzed, spray-dried, agglomerated oat powder by weight of the total drinkable food product. In some embodiments, the agglomerated oat powder has an average particle size of 150 to 450 μm. In some embodiments, at least 70% of the particles are within the range of 150 to 450 μm.

In a second aspect, U.S. Pat. No. 8,241,696 includes or can be modified to include a drinkable food product comprising milk and about 5 wt % to about 15 wt % hydrolyzed, spray-dried, agglomerated oat powder by weight of the total drinkable food product. In some embodiments, the agglomerated oat powder has an average particle size of 150 to 450 μm. In some embodiments, at least 70% of the particles are within the range of 150 to 450 μm.

In a third aspect, U.S. Pat. No. 8,241,696 includes or can be modified to include a drinkable oatmeal product comprising about 5 wt % to about 15 wt % hydrolyzed agglomerated oat flour by weight of the total drinkable food product; water; and a fruit component selected from the group consisting of fruit juice, yogurt containing fruit, fruit puree, fresh fruit, dried fruit powder, fruit preserves and combinations thereof. In some embodiments, the agglomerated oat powder has an average particle size of 150 to 450 μm. In some embodiments, at least 70% of the particles are within the range of 150 to 450 μm.

In a fourth aspect, U.S. Pat. No. 8,241,696 includes or can be modified to include a method of improving dispersability of oat powder in a beverage, comprising the steps of mixing about 5 wt % to about 15 wt % hydrolyzed, spray-dried, agglomerated oat powder with a liquid. In some embodiments, the agglomerated oat powder has an average particle size of 150 to 450 μm. In some embodiments, at least 70% of the particles are within the range of 150 to 450 μm.

U.S. patent application Ser. No. 12/264,399, entitled “Soluble Oat Flour and Method of Making Utilizing Enzymes,” was published as U.S. Patent Application Publication No. 2010/0112127 A1 and issued as U.S. Pat. No. 8,574,644, which are all hereby incorporated by reference in their entirety as examples. In one aspect, U.S. Pat. No. 8,574,644 includes or can be modified to include a method of producing a whole oat flour having soluble fiber comprising one or more steps selected from the following list of steps. A first step comprises combining a whole oat flour starting mixture and an α-amylase enzyme water solution to form a wetted enzyme starting mixture having a moisture content of about 25 to about 40 wt. %. A second step comprises heating the wetted enzyme starting mixture to between about 120° F. and about 200° F. A third step comprises adding the heated wetted mixture to an extender and extending for 1 to 1.5 minutes at a barrel temperature of about 140° F. to about 250° F. to form the whole oat flour having soluble fiber. In some embodiments, the temperature of the mixture increases in the extender to a temperature to deactivate the enzyme.

In a second aspect, U.S. Pat. No. 8,574,644 includes or can be modified to include a method for preparing a beverage containing a whole oat flour having soluble fiber comprising one or more steps selected from the following list of steps. A first step comprises combining a whole oat flour starting mixture and an α-amylase enzyme water solution to form wetted enzyme starting mixture having a moisture content of about 25 to about 40 wt. %. A second step comprises heating the wetted enzyme starting mixture to between about 120° F. and about 200° F. A third step comprises adding the heated wetted mixture to an extruder and extruding for 1 to 1.5 minutes at a barrel temperature of about 140° F. to about 250° F. to form the whole oat flour having soluble fiber. A fourth step comprises adding the whole oat flour having soluble fiber to a beverage. In some embodiments, the temperature of the mixture increases in the extruder to a temperature to deactivate the enzyme.

In a third aspect, U.S. Pat. No. 8,574,644 includes or can be modified to include a method for preparing a food product containing a whole oat flour having soluble fiber comprising one or more steps selected from the following list of steps. A first comprises combining a whole oat flour starting mixture and an α-amylase enzyme water solution to form a wetted enzyme starting mixture having a moisture content of about 25 to about 40 wt. %. A second step comprises heating the wetted enzyme starting mixture to between about 120° F. and about 200° F. A third step comprises adding the heated wetted mixture to an extruder and extruding for 1 to 1.5 minutes at a barrel temperature of about 140° F. to about 250° F. to form the whole oat flour having soluble fiber. A fourth step comprises adding the whole oat flour having soluble fiber to a mixture for a food product. In some embodiments, the temperature of the mixture increases in the extruder to a temperature to deactivate the enzyme.

U.S. patent application Ser. No. 12/264,404, entitled “Soluble Oat or Barley Flour and Method of Making Utilizing a Continuous Cooker,” was published as U.S. Patent Application Publication No. 2010/0112167 A1 and issued as U.S. Pat. No. 8,802,177, which are all hereby incorporated by reference in their entirety as examples. In one aspect, U.S. Pat. No. 8,802,177 includes or can be modified to include a method of producing a soluble whole oat or barley flour comprising one or more steps selected from the following list of steps. A first step comprises hydrating and heating to 140° F.-160° F. a whole oat or barley flour starting mixture to form a uniform free flowing wetted material having a moisture level of about 28 to about 30% by weight. In some embodiments, the whole oat or barley flour starting mixture comprises about 80 to about 95% by weight whole oat or barley flour, sugar, and at least one antioxidant. A second step comprises adding the hydrated whole oat or barley flour starting mixture to a low-shear extruder. In some embodiments, the extruder barrel temperature of about 140° F. to about 250° F. A third step comprises extruding the whole oat or barley flour starting mixture at a screw speed of 200 to 300 rpm to obtain a dough having a temperature of 212° F.-260° F. and to gelatinize and dextrinize the dough within the extruder. A fourth step comprises granulating the dough exiting the extruder to form the soluble whole oat or barley flour having a particle size of 50 to 250 microns.

In a second aspect, U.S. Pat. No. 8,802,177 includes or can be modified to include a method for preparing a beverage containing a soluble whole oat or barley flour comprising one or more steps selected from the following list of steps. A first step comprises hydrating and heating to 140° F.-160° F. a whole oat or barley flour starting mixture to form a uniform free flowing wetted material having a moisture level of about 28 to about 30% by weight. In some embodiments, the whole oat or barley flour starting mixture comprises about 80 to about 95% by weight whole oat or barley flour, sugar, and at least one antioxidant. A second step comprises adding the hydrated whole oat or barley flour starting mixture to a low-shear extruder. In some embodiments, the extruder barrel temperature of about 140° F. to about 250° F. A third step comprises extruding the whole oat or barley flour starting mixture and heat at a screw speed of 200 to 300 rpm to obtain a dough having a temperature of 212° F.-260° F., and to gelatinize and dextrinize the dough within the extruder. A fourth step comprises granulating the dough exiting the extruder to form the soluble oat or barley flour having a particle size of 50 to 250 microns. A fifth step comprises adding the soluble whole oat or barley flour to a beverage. In some embodiments, the soluble flour is added to provide a beverage having 1 to 25% by weight soluble fiber based on total weight of the beverage.

U.S. patent application Ser. No. 12/814,610, entitled “Method of Preparing Highly Dispersible Whole Grain Flour,” was published as U.S. Patent Application Publication No. 2010/0316765 A1 and issued as U.S. Pat. No. 8,586,113, which are all hereby incorporated by reference in their entirety as examples. In one aspect, U.S. Pat. No. 8,586,113 includes or can be modified to include a method of preparing a highly dispersible whole grain flour comprising one or more steps selected from the following list of steps. A first step comprises hydrolyzing a whole grain flour using alpha-amylase, the alpha-amylase hydrolyzes the whole grain flour while maintaining the integrity of the whole grain; and then optionally heating the hydrolyzed whole grain flour to temperature to deactivate the alpha-amylase. A second step comprises finely milling the hydrolyzed whole grain flour to a particle size of about 50-200 microns. A third step comprises agglomerating the whole grain flour.

In a second aspect, U.S. Pat. No. 8,586,113 includes or can be modified to include a method of preparing a highly dispersible whole grain flour comprising one or more steps selected from the following list of steps. A first step comprises combining a whole grain flour starting mixture and alpha-amylase to form an enzyme starting mixture. In some embodiments, the alpha-amylase hydrolyzes the whole grain flour while maintaining the integrity of the whole grain. A second step comprises introducing the enzyme starting mixture to an extruder. A third step comprises gelatinizing the whole grain flour by mechanical action and heating the extruder to form hydrolyzed whole grain flour dough, and optionally increasing the temperature of the dough in the extruder to a temperature to deactivate the enzyme. A fourth step comprises pelletizing the hydrolyzed whole grain flour dough to form hydrolyzed whole grain pellets. A fifth step comprises finely milling the hydrolyzed whole grain pellets to form hydrolyzed whole grain particles having a particle size of about 50-200 microns. A sixth step comprises agglomerating the hydrolyzed whole grain particles to form highly dispersible hydrolyzed whole grain flour.

U.S. patent application Ser. No. 12/666,509, entitled “Soluble Oat Flour and Method of Making Utilizing Enzymes,” was published as U.S. Patent Application Publication No. 2011/0189341 A1 and issued as U.S. Pat. No. 8,591,970, which are all hereby incorporated by reference in their entirety as examples. In one aspect, U.S. Pat. No. 8,591,970 is directed to a beverage containing a soluble whole oat flour. In some embodiments, the soluble whole oat flour is prepared by a method comprising one or more steps selected from the following list of steps. A first step comprises combining a whole oat flour starting mixture and an α-amylase enzyme water solution to form a wetted enzyme starting mixture having a moisture content of about 25 to about 40 wt. %. A second step comprises heating the wetted enzyme starting mixture to between about 120° F. and about 200° F. A third step comprises adding the heated wetted mixture to an extruder and extruding for 1 to 1.5 minutes and to form the soluble whole oat flour. In some embodiments, the temperature of the mixture increases in the extruder to a temperature to deactivate the enzyme.

U.S. patent application Ser. No. 12/666,506, entitled “Soluble Oat or Barley Flour and Method of Making Utilizing a Continuous Cooker,” was published as U.S. Patent Application Publication No. 2011/0281007 A1 and issued as U.S. Pat. No. 8,795,754, which are all hereby incorporated by reference in their entirety as examples. In one aspect, U.S. Pat. No. 8,795,754 includes or can be modified to include beverage comprising soluble whole oat or barley flour. In some embodiments, the beverage is prepared by a method comprising one or more steps selected from the following list of steps. A first step comprises hydrating and heating to 140° F.-160° F. a whole oat or barley flour starting mixture to form a uniform free flowing material having a moisture level of about 28 to about 30% by weight. In some embodiments, the whole oat or barley flour starting mixture comprises about 80 to about 95% by weight whole oat or barley flour, sugar, and at least one antioxidant. A second step comprises adding the hydrated whole oat or barley flour starting mixture to a low-shear extruder having an extruder barrel temperature of about 140° F. to about 250° F. A third step comprises extruding the whole oat or barley flour starting mixture at a screw speed of 200 to 300 rpm to obtain a dough having a temperature of 212° F.-260° F., and to gelatinize and dextrinize the dough within the extruder. A fourth step comprises granulating the dough exiting the extruder to form the soluble whole oat or barley flour having a particle size of 50 to 250 microns. A fifth step comprises adding the soluble whole oat or barley flour to a beverage to provide a beverage having 1 to 25% by weight soluble fiber based on total weight of the beverage.

U.S. patent application Ser. No. 13/547,733, entitled “Method of Preparing an Oat-Containing Dairy Beverage,” was published as U.S. Patent Application Publication No. 2013/0017300 A1 which are all hereby incorporated by reference in their entirety as examples. In one aspect, U.S. Patent Application Publication No. 2013/0017300 A1 includes or can be modified to include a ready-to-drink milk-based oat beverage comprising: a. hydrolyzed oat flour; b. fluid milk; c. at least one nutritive or non-nutritive sweetener; d. at least one stabilizer; e. at least one salt; and f a combination thereof. In some embodiment, the beverage has a shelf life of about 6 months at 25° C.

In a second aspect, U.S. Patent Application Publication No. 2013/0017300 A1 includes or can be modified to include a method for preparing an oat containing beverage comprising one or more steps selected from the following steps. A first step comprises hydrating hydrolyzed oat flour under ambient conditions or chilled conditions. A second step comprises introducing the hydrolyzed oat flour to chilled fluid milk at a temperature of about 4-7° C. to form a raw beverage. A third step comprises maintaining the raw beverage at a temperature of 4-7° C. A fourth step comprises preheating the raw beverage to 80° C. prior to homogenization. A fifth step comprises homogenizing the raw beverage to form a final beverage. A sixth step comprises introducing the final beverage to sterilization at a temperature of about 140-145° C.

In a third aspect, U.S. Patent Application Publication No. 2013/0017300 A1 includes or can be modified to include a system for preparing an oat containing beverage comprising several components selected from the group consisting of: a. an agitated vessel for hydrating hydrolyzed oat flour under ambient conditions; b. a vessel for storing chilled fluid milk at a temperature of about 4-7° C.; c. a mixer/disperser to mix the chilled fluid milk and hydrated hydrolyzed oat flour to form a raw beverage; d. a preheater to preheat the raw beverage; e. a homogenizer to form a final beverage from the raw beverage; f an aseptic sterilizer to form a final sterilized beverage from the final beverage, g. an aseptic filler/packaging to finalize shelf stable product ready to drink; and h. a combination thereof.

U.S. patent application Ser. No. 13/784,255, entitled “Method of Processing Oats to Achieve Oats with an Increased Avenanthramide Content,” was published as U.S. Patent Application Publication No. 2013/0183405 A1 and issued as U.S. Pat. No. 9,504,272, which are all hereby incorporated by reference in their entirety as examples. In one aspect, U.S. Pat. No. 9,504,272 includes or can be modified to include a composition comprising whole grain oat flour. In some embodiments, the whole grain oat flour meets the standard of identity for whole grain, the composition disperses in less than about 5 seconds in a liquid media at 25° C., the whole grain oat flour contains about 20-35% more avenanthramides on a weight basis compared to native whole grain oat flour, or a combination thereof.

In a second aspect, U.S. Pat. No. 9,504,272 includes or can be modified to include a composition comprising whole grain oat flour. In some embodiments, the whole grain oat flour contains about 20-35% more avenanthramides on a weight basis compared to native whole grain oat flour.

In a third aspect, U.S. Pat. No. 9,504,272 includes or can be modified to include a composition produced using a process comprising one or more steps selected from the following list of steps. A first step comprises combining a whole grain oat flour starting mixture with an aqueous enzyme solution to form an enzyme starting mixture having a moisture content of 25 to 40 wt %. A second step comprises heating the enzyme starting mixture to between about 120° F. and 200° F. A third step comprises adding the heated starting mixture to an extruder and extruding the mixture until the temperature of the mixture increases to about 260° F. to 300° F. In some embodiments, the enzyme is deactivated to form the composition, the composition comprises whole grain oat flour, the whole grain oat flour maintains its standard of identity throughout processing, the composition disperses in less than about 5 seconds in a liquid media at 25° C., the whole grain oat flour contains at least 20% higher level of avenanthramides on a weight basis compared to native whole grain oat flour, or a combination thereof.

U.S. patent application Ser. No. 13/833,717, entitled “Method of Preparing Highly Dispersible Whole Grain Flour with an Increased Avenanthramide Content,” was published as U.S. Patent Application Publication No. 2013/0209610 A1 and issued as U.S. Pat. No. 9,011,947, which are all hereby incorporated by reference in their entirety as examples. In one aspect, U.S. Pat. No. 9,011,947 includes or can be modified to include a highly dispersible whole grain oat flour containing about 20-35% more avenanthramides compared to native whole oat flour. In some embodiments, the whole grain oat flour is agglomerated following hydrolysis, pelletizing and milling.

In a second aspect, U.S. Pat. No. 9,011,947 includes or can be modified to include a highly dispersible whole grain oat flour produced using a process comprising one or more steps selected from the following list of steps. A first step comprises combining a native whole grain oat flour starting mixture with an aqueous enzyme solution to form an enzyme starting mixture having a moisture content of 25 to 40 wt %. A second step comprises heating the enzyme starting mixture. A third step comprises adding the heated starting mixture to an extruder and extruding the mixture until the temperature of the mixture increases to about 260° F. to 300° F. In some embodiments, the enzyme is deactivated. A fourth step comprises pelletizing the extruded flour. A fifth step comprises drying the pelletized extruded flour. A sixth step comprises milling the pelletized extruded flour to a particle size of about 50-420 microns. A seventh step comprises agglomerating the milled extruded flour to a particle size of about 150-1000 microns. In some embodiments, the highly dispersible whole grain oat flour contains at least 20% higher level of avenanthramides compared to native whole oat flour.

U.S. patent application Ser. No. 14/059,566, entitled “Soluble Oat Flour and Method of Making Utilizing Enzymes,” was published as U.S. Patent Application Publication No. 2014/0050819 A1 and issued as U.S. Pat. No. 9,149,060, which are all hereby incorporated by reference in their entirety as examples. In one aspect, U.S. Pat. No. 9,149,060 includes or can be modified to include a method of producing a whole oat flour having soluble fiber. In some embodiments, the method comprises one or more steps selected from the following list of steps. A first step comprises forming a whole oat flour starting mixture comprising about 50 to about 100% whole oat flour, 0 to about 15% granulated sugar, and 0 to about 15% maltodextrin. A second step comprises combining the whole oat flour starting mixture and an α-amylase enzyme water solution to form a wetted enzyme starting mixture having a moisture content of about 25 to about 40 wt. %. A third step comprises heating the wetted enzyme starting mixture to between about 120° F. and about 200° F. A fourth step comprises adding the heated wetted mixture to an extruder and extruding for 1 to 1.5 minutes to produce the whole oat flour having soluble fiber. In some embodiments, the temperature of the mixture increases in the extruder to a temperature to deactivate the enzyme.

In a second aspect, U.S. Pat. No. 9,149,060 includes or can be modified to include a method for producing a beverage containing a whole oat flour having soluble fiber. In some embodiments, the method comprises one or more steps selected from the following list of steps. A first step comprises forming a whole oat flour starting mixture comprising about 50 to about 100% whole oat or barley flour, 0 to about 15% granulated sugar, and 0 to about 15% maltodextrin. A second step comprises combining the whole oat flour starting mixture and an α-amylase enzyme water solution to form wetted enzyme starting mixture having a moisture content of about 25 to about 40 wt. %. A third step comprises heating the wetted enzyme starting mixture to between about 120° F. and about 200° F. A fourth step comprises adding the heated wetted mixture to an extruder and extruding for 1 to 1.5 minutes to form the whole oat flour having soluble fiber. In some embodiments, the temperature of the mixture increases in the extruder to a temperature to deactivate the enzyme. A fifth step comprises adding the whole oat flour having soluble fiber to a beverage.

U.S. patent application Ser. No. 14/209,000, entitled “Food Products Prepared with Soluble Whole Grain Oat Flour,” was published as U.S. Patent Application Publication No. 2014/0193564 A1 and issued as U.S. Pat. No. 9,510,614, which are all hereby incorporated by reference in their entirety as examples. In one aspect, U.S. Pat. No. 9,510,614 includes or can be modified to include a beverage comprising whole grain oat flour. In some embodiments, the whole grain oat flour is highly dispersible in water, the beverage provides ½ to 1 serving of whole grain per 8 oz serving of the beverage, the serving of whole grain is 16 g of whole grain, or a combination thereof. In some embodiments, the whole grain oat flour is produced by a process comprising one or more steps selected from the following list of steps. A first step comprises hydrolyzing starch in the whole grain oat flour in an extruder. In some embodiments, the starch hydrolysis is catalyzed by α-amylase. A second step comprises deactivating the a-amylase in the extruder before the starch hydrolysis results in a substantial change in a mass concentration of sugar in the whole grain oat flour.

In a second aspect, U.S. Pat. No. 9,510,614 includes or can be modified to include a semi-solid dairy product comprising whole grain oat flour in an amount of 2 to 11 wt. % based on total weight of the semi-solid dairy product. In some embodiments, the whole grain oat flour is highly dispersible in water. In some embodiments, the whole grain oat flour is produced by a process comprising one or more steps selected from the following list of steps. A first step comprises hydrolyzing starch in the whole grain oat flour in an extruder. In some embodiments, the starch hydrolysis is catalyzed by α-amylase. A second step comprises deactivating the α-amylase in the extruder before the starch hydrolysis results in a substantial change in a mass concentration of sugar in the whole grain oat flour.

In a third aspect, U.S. Pat. No. 9,510,614 includes or can be modified to include an instant powder for preparing a cold beverage comprising 25 to 60 wt. % whole grain oat flour. In some embodiments, the whole grain oat flour is highly dispersible in water; when the whole grain oat flour is hydrated in liquid to form the beverage, the beverage provides ½ to 1 serving of whole grain per 8 oz serving of the beverage; the serving of whole grain is 16 g of whole grain, or a combination thereof. In some embodiments, the whole grain oat flour is produced by a process comprising one or more steps selected from the following list of steps. A first step comprises hydrolyzing starch in the whole grain oat flour in an extruder. In some embodiments, the starch hydrolysis is catalyzed by α-amylase. A second step comprises deactivating the α-amylase in the extruder before the starch hydrolysis results in a substantial change in a mass concentration of sugar in the whole grain oat flour.

In a fourth aspect, U.S. Pat. No. 9,510,614 includes or can be modified to include an instant powder comprising 25 to 35 wt. % whole grain oat flour. In some embodiments, the whole grain oat flour is highly dispersible in water. In some embodiments, when hydrated in liquid to provide a product, the powder provides ½ to 1 whole serving of whole grain per 4 to 8 oz serving of the product; and/or the serving of whole grain is 16 g of whole grain. In some embodiments, the whole grain oat flour is produced by a process comprising one or more steps selected from the following list of steps. A first step comprises hydrolyzing starch in the whole grain oat flour in an extruder. In some embodiments, the starch hydrolysis is catalyzed by α-amylase. A second step comprises deactivating the α-amylase in the extruder before the starch hydrolysis results in a substantial change in a mass concentration of sugar in the whole grain oat flour.

U.S. patent application Ser. No. 14/209,075, entitled “Food Products Prepared with Soluble Whole Grain Oat Flour,” was published as U.S. Patent Application Publication No. 2014/0193563 A1 and issued as U.S. Pat. No. 9,622,500, which are all hereby incorporated by reference in their entirety as examples. In one aspect, U.S. Pat. No. 9,622,500 includes or can be modified to include a bakery product selected from the group consisting of muffins, cookies, breads, bagels, pizza crust, cakes, crepes, and pancakes. In some embodiments the bakery product is prepared from ingredients comprising whole grain oat flour in an amount of 2 to 10 wt. % as a texturizer. In some embodiments, the whole grain oat flour is highly dispersible in water so that there are no lumps of the whole grain oat flour in a mixture of the whole grain oat flour and water at 25° C. after stirring the mixture for 5 seconds.

U.S. patent application Ser. No. 14/959,941, entitled “Whole Grain Composition Comprising Hydrolyzed Starch,” was published as U.S. Patent Application Publication No. 2016/0081375 A1, which are all hereby incorporated by reference in their entirety as examples. In one aspect, U.S. Patent Application Publication No. 2016/0081375 includes or can be modified to include a composition comprising a whole grain, and the whole grain comprises hydrolyzed starch.

U.S. patent application Ser. No. 15/077,670, entitled “Method, Apparatus, and Product Providing Hydrolyzed Starch and Fiber,” which is hereby incorporated by reference in its entirety as an example. In one aspect, U.S. patent application Ser. No. 15/077,670 includes or can be modified to include a composition comprising at least one material selected from the group consisting of at least a portion of grain and at least a portion of pulse. In some embodiments, the at least one material comprises hydrolyzed starch and hydrolyzed fiber; the hydrolyzed starch consists of starch molecules; the average molecular weight of the hydrolyzed starch molecules in the composition is a first fraction of the molecular weight of unhydrolyzed starch molecules; the unhydrolyzed starch molecules are equivalent in kind and condition to the hydrolyzed starch molecules, except that the unhydrolyzed starch molecules have not been hydrolyzed; the first fraction is no more than about 0.80; the hydrolyzed fiber consists of fiber molecules; the average molecular weight of the hydrolyzed fiber molecules in the composition is a second fraction of the molecular weight of unhydrolyzed fiber molecules; the unhydrolyzed fiber molecules are equivalent in kind and condition to the hydrolyzed fiber molecules, except that the unhydrolyzed fiber molecules have not been hydrolyzed; the second fraction is no more than about 0.80; or a combination thereof.

In a second aspect, U.S. patent application Ser. No. 15/077,670 includes or can be modified to include a method comprising one or more steps selected from the following list of steps. A first step comprises providing starting components comprising a first enzyme; a second enzyme; water; and a starting composition. In some embodiments, the starting composition comprises at least one material selected from the group consisting of at least a portion of grain and at least a portion of pulse. In some embodiments, the at least one material comprises starch and fiber. A second step comprises hydrolyzing the fiber in the at least one material through a fiber hydrolysis reaction. In some embodiments, the fiber hydrolysis reaction is catalyzed by the first enzyme. A third step comprises hydrolyzing the starch in the at least one material through a starch hydrolysis reaction. In some embodiments, the starch hydrolysis reaction is catalyzed by the second enzyme. A fourth step comprises deactivating the first enzyme. A fifth step comprises deactivating the second enzyme. In some embodiments the method provides a product composition.

U.S. patent application Ser. No. 15/077,676, entitled “Method and Apparatus for Controlled Hydrolysis,” which is hereby incorporated by reference in its entirety as an example. In one aspect, U.S. patent application Ser. No. 15/077,676 includes or can be modified to include a method comprising one or more steps selected from the following list of steps. A first step comprises hydrolyzing a first reagent in a first hydrolysis reaction. A second step comprises deactivating a first enzyme catalyzing the first hydrolysis reaction. In some embodiments, the deactivating step lasts no more than about 10 seconds.

In a second aspect, U.S. patent application Ser. No. 15/077,670 includes or can be modified to include a hydrolysis reactor comprising a conduit; a composition inlet in the conduit for a composition; a first enzyme inlet in the conduit downstream of the composition inlet; a first deactivating mechanism downstream of the first enzyme inlet to deactivate the first enzyme; or a combination thereof.

U.S. patent application Ser. No. 15/077,75800, entitled “Method and Composition Comprising Hydrolyzed Starch,” was published as U.S. Patent Application Publication No. 2016/0198754 A1, which are all hereby incorporated by reference in their entirety as examples. In one aspect, U.S. Patent Publication No. 2016/0198754 A1 includes or can be modified to include a method comprising one or more steps selected from the following list of steps. A first step comprises combining at least a portion of pulse and a suitable enzyme to form an enzyme-pulse starting mixture. In some embodiments, the enzyme-pulse starting mixture comprises starch. A second step comprises heating the enzyme-pulse starting mixture to between about 48.89° C. and about 93.33° C. to begin to hydrolyze the starch, thereby providing a heated pulse mixture. A third step comprises extruding the heated pulse mixture to continue hydrolyzing the starch and further to gelatinize and cook the heated pulse mixture thereby providing a pulse product comprising gelatinized, hydrolyzed starch.

In a second aspect, U.S. Patent Publication No. 2016/0198754 A1 includes or can be modified to include a composition comprising at least a portion of pulse, and the at least a portion of pulse comprises gelatinized, hydrolyzed starch.

In a third aspect, U.S. Patent Publication No. 2016/0198754 A1 includes or can be modified to include a composition comprising whole grain, and the whole grain comprises gelatinized, hydrolyzed starch.

U.S. patent application Ser. No. 15/481,286, entitled “Food Products Prepared with Soluble Whole Grain Oat Flour,” which is hereby incorporated by reference in its entirety as an example. In one aspect, U.S. patent application Ser. No. 15/481,286 includes or can be modified to include instant oatmeal comprising oat flakes and a powder. In some embodiments, the powder comprises flavors, sweeteners, and at least one texturizer. In some embodiments, the at least one texturizer comprises 0.09 to 0.3 wt. % whole grain oat flour; and/or the whole grain oat flour is highly dispersible in water so that there are no lumps of the whole grain oat flour in a mixture of the whole grain oat flour and water at 25° C. after stirring the mixture for 5 seconds.

In a second aspect, U.S. patent application Ser. No. 15/481,286 includes or can be modified to include a ready-to-eat soup comprising 2 to 10 wt. % of whole grain oat flour based on total weight of the soup. In some embodiments, the whole grain oat flour provides at least ½ serving of whole grains per 8 oz serving; and/or the whole grain oat flour is highly dispersible in water so that there are no lumps of the whole grain oat flour in a mixture of the whole grain oat flour and water at 25° C. after stirring the mixture for 5 seconds.

In a third aspect, U.S. patent application Ser. No. 15/481,286 includes or can be modified to include a frozen commodity selected from the group consisting of ice cream and slushies. In some embodiments, the frozen commodity comprises whole grain oat flour in an amount of 2 to 10 wt. % based on total weight of the frozen commodity; and/or the whole grain oat flour is highly dispersible in water so that there are no lumps of the whole grain oat flour in a mixture of the whole grain oat flour and water at 25° C. after stirring the mixture for 5 seconds.

U.S. patent application Ser. No. 12/951,950, entitled “Thick Juice Beverages,” was published as U.S. Patent Application Publication No. 2011/0129591 A1 and issued as U.S. Pat. No. 8,673,382, which are all hereby incorporated by reference in their entirety as examples. In one aspect, U.S. Pat. No. 8,673,382 includes or can be modified to include a beverage that includes a base juice and also homogenized pulp. In some embodiments, the homogenized pulp is included in an amount between about 15%-45% by weight, and has particles size between about 40 microns-700 microns in diameter. In some embodiments, the measured viscosity of the beverage is between about 50 cps-125 cps at the time of manufacture and the beverage exhibits both a smooth mouthfeel and a taste profile that are not significantly different from that of the base juice.

In a second aspect, U.S. Pat. No. 8,673,382 includes or can be modified to include a beverage that includes a base juice and homogenized finisher-derived solids. In some embodiments, the finisher-derived solids are included in an amount between about 15%-40% by weight, and have particle sizes between about 40 microns-1400 microns in diameter. In some embodiments, the measured viscosity of the beverage is between about 50 cps-125 cps at the time of manufacture and the beverage exhibits both a smooth mouthfeel and a taste profile that are not significantly different from that of the base juice.

In a third aspect, U.S. Pat. No. 8,673,382 includes or can be modified to include a beverage that includes homogenized finisher-derived solids having a particle size between about 40 microns-1500 microns in diameter, and homogenized pulp having a particle size between about 40 microns-750 microns in diameter. In some embodiments, the beverage has a measured viscosity between about 50 cps-125 cps at the time of manufacture and the beverage exhibits a taste profile that is not significantly different from that of the base juice.

In a fourth aspect, U.S. Pat. No. 8,673,382 includes or can be modified to include a beverage consisting essentially of a base juice and homogenized pulp in an amount between about 15%-45% by weight, with particle sizes of between about 40 microns-700 microns in diameter. In some embodiments, the beverage has a measured viscosity between about 50 cps-125 cps at the time of manufacture and the beverage exhibits both a smooth mouthfeel and a taste profile that are not significantly different from that of the base juice.

U.S. patent application Ser. No. 13/249,289, entitled “Processing of Whole Fruits and Vegetables, Processing of Side-Stream Ingredients of Fruits and Vegetables, and Use of the Processed Fruits and Vegetables in Beverage and Food Products,” was published as U.S. Patent Application Publication No. 2012/0088015 A1, which are all hereby incorporated by reference in their entirety as examples. In one aspect, U.S. Patent Application Publication No. 2012/0088015 includes or can be modified to include a fruit or vegetable processed product that includes pomace or at least one whole fruit or vegetable. In some embodiments, the processed product has a particle size less than 250 microns.

In a second aspect, U.S. Patent Application Publication No. 2012/0088015 includes or can be modified to include a beverage that includes water and a fruit or vegetable processed product comprising pomace or at least one whole fruit or vegetable. In some embodiments, the processed product has a particle size less than 250 microns.

In a third aspect, U.S. Patent Application Publication No. 2012/0088015 includes or can be modified to include a method of processing pomace that includes the step of reducing a particle size of the pomace to less than 250 microns.

In a fourth aspect, U.S. Patent Application Publication No. 2012/0088015 includes or can be modified to include a method of treating at least one whole fruit or vegetable that includes the step of processing the whole fruits or vegetables to provide a product having a particle size of less than 250 microns.

In a fifth aspect, U.S. Patent Application Publication No. 2012/0088015 includes or can be modified to include a method of improving the dispersability of pomace in beverages that includes the step of reducing the particle size of the pomace to less than 250 microns prior to adding to the beverage.

In a sixth aspect, U.S. Patent Application Publication No. 2012/0088015 includes or can be modified to include a method of testing the fiber content of pomace comprising heating the pomace up to 100° C. for a time sufficient for enzyme inactivation and then subjecting the pomace to AOAC analysis.

In another aspect, U.S. Patent Application Publication No. 2012/0088015 includes or can be modified to include a method of processing pomace that includes the steps of obtaining a pomace press cake by extracting juice from a fruit, vegetable, or a combination of fruit and vegetable; hydrating the pomace press cake; acidifying the pomace press cake with an organic acid; and micro-grinding the hydrated, acidified pomace press cake to reduce the particle size of the pomace to less than 250 microns.

U.S. patent application Ser. No. 13/305,360, entitled “Fiber Obtained from Fruit or Vegetable Byproducts,” was published as U.S. Patent Application Publication No. 2012/0135109 A1, which are all hereby incorporated by reference in their entirety as examples. In one aspect, U.S. Application Publication No. 2012/0135109 includes or can be modified to include a fiber extracted from a fruit or vegetable byproduct. In some embodiments, the fiber, which has a molecular weight of between about 5000 g/mol-8000 g/mol, is extracted using a physical method to break the byproduct cell walls and enzymatic hydrolysis.

In a second aspect, U.S. Patent Application Publication No. 2012/0135109 includes or can be modified to include a fiber extracted from a fruit or vegetable byproduct. In some embodiments, the fiber, which has a molecular weight of between about 5000 g/mol-8000 g/mol, is extracted using at least one physical method to break the byproduct cell walls.

In a third aspect, U.S. Patent Application Publication No. 2012/0135109 includes or can be modified to include a pectic oligosaccharide extracted from a fruit or vegetable byproduct. In some embodiments, the pectic oligosaccharide has a molecular weight between about 300 g/mol-2500 g/mol.

In a fourth aspect, U.S. Patent Application Publication No. 2012/0135109 includes or can be modified to include a method for producing a soluble fiber including one or more of the following steps: reducing the particle size of a fruit or vegetable byproduct; subjecting the byproduct particles to a physical process to break cell walls of the byproduct particles; adding one or more enzymes to the byproduct particles; mixing or agitating the byproduct particles; and filtering the byproduct particles to provide a retentate and a permeate, the latter containing the soluble fiber.

In a fifth aspect, U.S. Patent Application Publication No. 2012/0135109 includes or can be modified to include a comestible that includes fiber extracted from a fruit or vegetable byproduct. In some embodiments, the fiber, which has a molecular weight of between about 5000 g/mol-8000 g/mol, is extracted by subjecting the fruit or vegetable byproduct to a physical process.

In a sixth aspect, U.S. Patent Application Publication No. 2012/0135109 includes or can be modified to include a comestible that includes pectic oligosaccharide extracted from a fruit or vegetable byproduct. In some embodiments, the pectic oligosaccharide has a molecular weight between about 300 g/mol-2500 g/mol.

U.S. patent application Ser. No. 14/262,213, entitled “Preparation and Incorporation of Co-Products into Beverages to Achieve Metabolic and Gut Health Benefits,” was published as U.S. Patent Application Publication No. 2014/0234476 A1, which are all hereby incorporated by reference in their entirety as examples. In one aspect, U.S. Patent Application Publication No. 2014/0234476 includes or can be modified to include a beverage that includes a liquid and a co-product from juice extraction. In some embodiments, the co-product has a number average particle size of between 0.1 microns-2000 microns, a total polyphenol content of at least 2500 parts per million, a moisture content of between 70%-85% by weight, and a combined peel and seed content between 0.01%-20% by weight. Consumption of the beverage by an individual confers a metabolic health benefit to the individual relative to a beverage composition not including the co-product.

In a second aspect, U.S. Patent Application Publication No. 2014/0234476 includes or can be modified to include a method for enhancing the metabolic and gut health of an individual which includes the step of administering a beverage composition comprising a liquid and a co-product from juice extraction to the individual. In some embodiments, the co-product has a number average particle size of between 0.1 microns-2000 microns, a total polyphenol content of at least 2500 parts per million, a moisture content of between 70%-85% by weight, and a combined peel and seed content between 0.01%-20% by weight. Additionally, the beverage has a viscosity between about 300 cps-3000 cps as measured using a Brookfield viscometer at 20 degrees Celsius.

In another aspect, U.S. Patent Application Publication No. 2014/0234476 includes or can be modified to include a beverage that includes a liquid and a co-product from juice extraction. In some embodiments, the co-product has a number average particle size of between 0.1 microns-2000 microns, a total polyphenol content of at least 2500 parts per million, and a combined peel and seed content between 0.01%-20% by weight. In addition, the beverage includes at least 10 wt % of the co-product, at least 2.5 grams of fiber per 8 ounce serving of the beverage, and a viscosity that is at least 1.5 times higher than a beverage composition not including the co-product. Moreover, the beverage confers a metabolic and gut health benefit to a consumer relative to the beverage composition not including the co-product.

U.S. patent application Ser. No. 14/766,828, entitled “Preparation and Incorporation of Co-Products into Beverages to Enhance Nutrition and Sensory Attributes,” was published as U.S. Patent Application Publication No. 2016/0000130 A1, which are all hereby incorporated by reference in their entirety as examples. In one aspect, U.S. Patent Application Publication No. 2016/0000130 includes or can be modified to include a beverage that includes juice and a co-product from juice extraction. In some embodiments, the co-product has a number average particle size of between 0.1 microns-2000 microns, a total polyphenol content of at least 2500 parts per million, a moisture content of between 70%-85% by weight, and a combined peel and seed content between 0.01%-20% by weight.

In a second aspect, U.S. Patent Application Publication No. 2016/0000130 includes or can be modified to include a beverage that includes juice in an amount between 5%-90% by weight; added water; at least one non-nutritive sweetener; at least one flavor; and a co-product from juice extraction. In some embodiments, the co-product has a number average particle size of between 0.1 microns-2000 microns, a total polyphenol content of at least 2500 parts per million, a moisture content of between 70%-85% by weight, and a combined peel and seed content between 0.01%-20% by weight. In addition, the beverage has a brix of between about 5 brix-9 brix.

In a third aspect, U.S. Patent Application Publication No. 2016/0000130 includes or can be modified to include a beverage including water; at least one sweetener; at least one acidulant; at least one flavor; at least one colorant; and a co-product from juice extraction. In some embodiments, the co-product has a number average particle size of between 0.1-2000 microns, a total polyphenol content of at least 2500 parts per million, a moisture content of between 70%-85% by weight, and a combined peel and seed content between 0.01%-20% by weight.

U.S. patent application Ser. No. 15/247,411, entitled “Viscosity Reduction of Beverages and Foods Containing High Fiber Fruit and Vegetable Materials,” was published as U.S. Patent Application Publication No. 2017/0055550 A1, which are all hereby incorporated by reference in their entirety as examples. In one aspect, U.S. Patent Application Publication No. 2017/0055550 includes or can be modified to include a beverage product that includes liquid and about 1-40 wt % enzymatically-treated pomace. In some embodiments, the enzymatically-treated pomace is derived from pomace selected from a group consisting of at least one fruit, at least one vegetable, and combinations thereof. In addition, the enzymatically-treated pomace has an amount of fiber that is the same before and after enzymatic treatment.

In a second aspect, U.S. Patent Application Publication No. 2017/0055550 includes or can be modified to include a food product that includes about 1-40 wt % enzymatically-treated pomace. In some embodiments, the enzymatically-treated pomace is derived from the group consisting of at least one fruit, at least one vegetable, and combinations thereof. In addition, the amount of fiber in the pomace remains the same before and after enzymatic treatment. In some embodiments, the food product exhibits a microbial shelf stability of 6 months.

In a third aspect, U.S. Patent Application Publication No. 2017/0055550 includes or can be modified to include a method that includes the steps of subjecting pomace to at least one enzyme to form a pomace-enzyme mixture. In some embodiments, the pomace includes fiber and the pomace-enzyme mixture includes the at least one enzyme in an amount between 0.15-1.0 wt % of the pomace. In some embodiments, the method also includes the steps of heating the pomace-enzyme mixture to 25-57° C. for 10-60 minutes, and deactivating the at least one enzyme to form the enzymatically-treated pomace.

U.S. patent application Ser. No. 15/394,949, entitled “Preparation and Incorporation of Co-Products into Beverages to Achieve Metabolic and Gut Health Benefits,” is hereby incorporated by reference in its entirety as an example. In one aspect, U.S. patent application Ser. No. 15/394,949 includes or can be modified to include a beverage that includes a liquid and a co-product formed from a pomace resulting from juice extraction. The co-product further includes phytonutrients from the pomace; a number average particle size between 0.1 microns-2000 microns, a peel and seed content between 0.01% and 80% by weight, and dietary fiber.

### Example 1

An illustrative method for making hydrolyzed, fermented plant origin material will now be described below. As an example, the plant-origin material can be a whole grain or a whole oat composition, in particular.

As a first step, the starting mixture and enzyme solution can be mixed in any suitable vessel, for example, a high speed mixer that permits liquid to be added to free-flowing flour. In some embodiments, the suitable vessel is called a preconditioner. The output is a free-flowing wetted flour mixture having a moisture content of about 25 to about 40%. The residence time is the time sufficient to obtain the desired result and typically 1 to 5 min.

As a second step, the free-flowing wetted flour mixture can be added to an extruder (continuous cooker) to gelatinize, hydrolyze, and cook the starch. The material can be heated from an initial inlet temperature to a final exit temperature in order to provide the energy for starch gelatinization. Given a plant-origin material for which the conversion of starch to non-starch components is undesirable (e.g., a whole grain), the flour mixture can reside in the extruder for a time sufficient to gelatinize and cook the starch in the flour mixture, but not long enough to substantially dextrinize or otherwise modify the starch to void the whole grain aspect of a whole grain plant-origin material, for example, at least 30 seconds or at least 1 minute, about 30 seconds to about 1.5 minutes or about 1 to about 1.5 minutes, to form a dough.

Starch gelatinization requires adequate water and energy (e.g., heat). As an example, the gelatinization temperature range for grains (e.g., oats, barley, wheat, etc.) is 127° F. to 160° F. (53-71° C.), or 127° F. to 138° F. (53-59° C.). If the moisture is less than about 60% then higher temperatures can be required, as illustrated by the higher temperatures used below in conjunction with a moisture content of about 25 to 40 wt. %. Additionally, it is worthwhile to note that in some embodiments, if the moisture content is above about 40 or 50 wt. %, an enzyme-catalyzed hydrolysis reaction that hydrolyzes starch can proceed so quickly that it must be closely controlled if the significant conversion of starch to non-starch components is undesirable or if the maintenance of a whole grain status or some other health benefit or claim is desired.

Heat can be applied through the extruder barrel wall such as with a jacket around the barrel through which a hot medium like steam, water or oil is circulated, or electric heaters imbedded in the barrel. Typically the extrusion occurs at barrel temperatures between 140° F. (60° C.) and 350° F. (176.67° C.), for example between 175° F. (79.44° C.) and 340° F. (171.11° C.), about 180° F. (82.22° C.) −300° F. (148.89° C.), or about 270° F. (132.22° C.) to about 310° F. (154.44°), or about 290° F. (143.33° C.). In some embodiments, the extrusion occurs at barrel temperatures between 140° F. (60° C.) and 300° F. (148.89° C.), or between 140° F. (60° C.) and 250° F. (121.11° C.). For example, in one embodiment, the wall temperature of the extruder barrel at the end of the extruder is about 280° F. (137.78° C.) to 300° F. (148.89° C.), or about 290° F. (143.33° C.), which can be useful to ensure that a hydrolysis-catalyzing enzyme is deactivated. Although, after reading this disclosure, a person skilled in the art would recognize that enzymes (e.g., amylases or cellulases) can be deactivated at different temperatures depending on which type of amylase or cellulase is used. Additionally, in some embodiments, the dough (e.g., in the extruder) is provided at a temperature that is approximately between 212° F. (100° C.) and 260° F. (126.67° C.).

Heat is also generated within the material by friction as it moves within the extruder by the dissipation of mechanical energy in the extruder, which is equal to the product of the viscosity and the shear rate squared for a Newtonian fluid. Shear is controlled by the design of the extruder screw(s) and the screw speed. Viscosity is a function of starch structure, temperature, moisture content, fat content and shear. The temperature of the dough increases in the extruder to about 212° F. (100° C.) to 350° F. (176.67° C.) or about 212° F. (100° C.) to 300° F. (148.89° C.). Although, in some embodiments, the dough temperatures are approximately between 212° F. (100° C.) and 260° F. (126.67° C.).

Extrusion conditions are chosen to adequately heat the extrudate to the desired temperature at the desired moisture content. Excessive cooked flavor (e.g., cooked grain flavor) can be generated if the combination of time and temperature of the extrudate exceeds an optimum combination of time and temperature. For some embodiments the moisture content of the extrudate is about 28% to about 33% with a wall temperature after the final barrel section is about 280° F. (137.78° C.) to about 330° F. (165.56° C.) or about 280° F. (137.78° C.) to about 305° F. (151.67° C.). Inadequate water addition can result in dextrinization of the starch in the extrudate. For example, in one embodiment, low shear is applied to the mixture in the extruder. In some embodiments (e.g., where the enzyme has preconditioned the starch), high shear is not required. Additionally, in some embodiments, high shear makes it difficult to control the degree of hydrolysis. It can also increase the dough temperature excessively, which can overcook it resulting in too much cooked flavor. As another example, high shear can dextrinize the starch, which can be undesirable in some embodiments. It is noted that the barrel temperature and the dough temperature can be different.

In some embodiments, the process balances limiting the dough temperature to avoid too much cooked flavor and to keep the enzyme active. For example, the process can be balanced such that the dough temperature rises to a sufficient temperature to deactivate the enzyme after a desired amount of hydrolysis has occurred. Depending on the enzyme used, sufficient temperatures to deactivate the enzyme can be generally 212° F. (100° C.) to about 330° F. (165.56° C.), or about 212° F. (100° C.) to 300° F. (148.89° C.), and/or at least 280° F. (137.78° C.). A low shear extrusion process is characterized relative to a high shear extrusion process by higher moisture and a lower shear screw design versus lower moisture and a higher shear screw design.

Any suitable extruder can be used, including suitable single screw or twin screw extruders. Typical, but not limiting, screw speeds are 200-350 rpm (e.g., 200-300 rpm).

The resulting product can be pelletized using a forming extruder and dried, for example, to about 1.5 to about 12%, about 1.5 to about 10%, or 6.5 to 8.5% moisture content by weight. The pellets can be granulated to a limited extent so that no more than 5 wt. % (i.e., 0 to 5 wt. %) of the granulated pellets pass through a US 40 screen. For example, the particle size of the resulting granulated product or flour can be about 1-500 microns, about 10-500 microns, about 1-450 microns, or about 30-420 microns. Although, in some embodiments, the pellets are granulated to a limited extent so that no more than 85 wt. % (i.e., 0 to 85 wt. %) of the particles pass through a US 30 screen. Additionally, in some embodiments, filters and/or screen can be used so that 90 to 100 wt. % of particles pass through a 500, 450 or 420 micron filter or screen and optionally are retained by a nominal 1, 10 or 30 micron filter or screen.

Jet milling can be used to mill the pellets produced in accordance with aspects of the present disclosure. Jet milling creates ultrafine particles. In particular, jet milling can reduce the particle size of all or much of (e.g., 90 to 100 wt. % of) the pelletized hydrolyzed plant-origin material flour (e.g., grain, oat, barley, or wheat flour) to less than or equal to about 90 microns, about 50 microns, or about 46 microns and greater than 0 microns. As one of ordinary skill in the art would recognize, alternative milling processes can be used to reduce the particle size or micronize the flour to, 0.5-50 microns, such as between 10 to 50 microns. For example, a milling process can be used to reduce the particle size of the flour so that 90 to 100 wt. % of the flour passes through a nominal 90, 50, or 46 micron filter or screen and optionally is retained by a nominal 0.5, 1, or 10 micron filter or screen.

The resulting hydrolyzed plant-origin material flour (e.g., oat flour) can include beta-glucan soluble fiber, such as beta-1, 3-glucan, beta-1, 6-glucan, or beta-1, 4-glucan or mixtures thereof. In addition to beta-glucan naturally present in the hydrolyzed plant-origin material (e.g., oats), beta-glucan can also be added as approved by the FDA. In certain embodiments, the hydrolyzed plant-origin material (e.g., oat flour) preferably contains at least about 3%, at least about 4%, or about 3% to 5% or about 3.7% to 4% beta-glucan on a dry weight basis. In certain embodiments, a liquid, semi-solid, or solid product including the hydrolyzed plant-origin material flour (e.g., oat flour) contains 0.1% to about 1.5% beta-glucan, or about 0.8% to 1.3% beta-glucan. Other amounts of beta-glucan are also useful. Additionally, in some embodiments, the hydrolyzed plant-origin material (e.g., oat flour) can contain at least about 8%, 9%, or 10% or about 8% to about 12% total dietary fiber by weight. Furthermore, for example, in accordance with 21 CFR 101.81, a whole oat flour can be produced from 100 percent dehulled, clean oat groats by steaming and grinding, such that there is no significant loss of oat bran in the final flour, the final flour provides at least 4% beta-glucan on a dry weight basis, and the final flour provides at least 10% total dietary fiber on a dry weight basis.

Hydrolyzed plant-origin material made using the method described above can be used to make the fermented plant-origin material described herein. For example, 12 wt. % Solu-Morrison flour obtained from the method described above, can be combined with 2 wt. sucrose and 86 wt. % water to provide a starting oat slurry. For example, the starting oat slurry can have a viscosity of about 1500 to 2000 cP at 38° C. The starting oat slurry can be pumped into a vessel with a modified impeller and fermentation culture can be added to provide a fermentation slurry comprising 99.98 wt. % starting oat slurry (e.g., 15 L) and 0.02 wt. % fermentation culture (e.g., 3 mL), which can comprise 5 different lacto-bacillus strands. An example of a fermentation culture is a lactic acid fermentation culture available from Chr Hansen of Hoersholm, Denmark, for example, YoFlex® (e.g., YF-L02 DA). The fermentation slurry can then be agitated at about at least 150 rpm for about 10 to 21 hours, at about 35 to 42° C., and at about atmospheric pressure. Once the agitation is complete, the fermented plant-origin material can be provided with a pH of below 4.5, 4.2, 4.0, 3.9 or 3.8, and optionally down to about 2.0, 2.1, 2.2, 2.3, 2.4 or 2.5. As an example, the pH can be lowered as a result of the production of lactic acid, which can be caused by a reaction between water and sugar, which can come from added sugar or sugar in or derived from the-plant origin material. In some embodiments, the fermented plant origin material comprises a titratable acidity of about 0.3 to about 0.4 wt. %. In one example, the fermentation slurry can be agitated for about 15 to 21 hours, at about 40° C. For example, the fermented plant-origin material can have a pH of below 4.0. The resulting viscosity of the fermented plant-origin material can be from about 5000 to 7000 cP at 25° C. The fermented plan-origin material can be incorporated into a drink, a food, or a spoonable product.

It should be understood that a product or composition described herein can take various forms and provide corresponding advantages in its various forms. One way that the form of a composition can be controlled is by varying the liquid content of the composition. For example, in some embodiments, the composition comprises a liquid mass concentration equal to 0-99%, 5-95%, 10-90%, 20-80%, 30-70%, 40-60%, 0-5%, 5-10%, 10-20%, 20-30%, 30-40%, 40-50%, 50-60%, 60-70%, 70-80%, 80-90%, 90-95%, 95-99%, or a combination thereof. Accordingly, the composition can be provided in the form of a flowable product, a liquid, a beverage, a semi-liquid, a solid, or a combination thereof. Additionally, the composition can have a viscosity equal to 0.5 to 3000, 0.5 to 2500, 0.5 to 2000, 0.5 to 1500, 0.5 to 1000, 0.5 to 800, 0.5 to 700, 0.5 to 600, 0.5 to 500, 0.5 to 400, 0.5 to 300, 0.5 to 250, 0.5 to 200, 0.5 to 150, 0.5 to 100, 1 to 100, 0.5 to 50, 1 to 50, 0.5 to 30, or 1 to 30 cP at 25° C. or at a desired consumption temperature, which can be useful when the product is intended to function as a beverage. A product (food product, beverage, semi-solid food, semi-liquid food, soup, texturizer/texture modifier, dip, or a combination thereof) can also be provided with a viscosity range whose endpoints are selected from any of the endpoints of the viscosity ranges listed herein. For example, for a smoothie, a higher viscosity range could be desirable. Meanwhile, for a juice, a viscosity closer to 1 cP could be desirable. Accordingly, in some embodiments, the method and/or product of the invention disclosed herein is versatile in the sense of providing a relatively higher degree of control over the viscosity of a product compared to alternatives that might provide otherwise similar advantages, for example, health benefits, nutrients, organoleptic properties, or a combination thereof.

Of course, the properties of a composition can also be controlled by selecting a specific type of liquid to include in the composition. Accordingly, in some embodiments, the composition comprises a liquid selected from the group consisting of water, milk, a dairy milk, a non-dairy milk, a vegetable juice, a fruit juice, and a combination thereof.

Embodiments of a product or composition as described herein can have various useful functions. For example, in some embodiments the composition is a food product 0456.

In some embodiments, the composition is a prebiotic.

In some embodiments, the composition is a glycemic index reducer that reduces the glycemic index of a food, for example, it is contemplated that the glycemic index could be reduced by at least 1, 2, 3, 4, 5, 6, 7, 8, 9, 10, 15, 20, 25 or 30%. In some embodiments, the composition is a glycemic index reducer that reduces the glycemic index of a food to which the glycemic index reducer is added by at least 5, 10, 15, 20, 25, 30, 40 or 50% or by no more than 10, 15, 20, 25, 30, 40, 50 or 60%, or a combination thereof. Additionally, in some embodiments, the invention can provide a food comprising a composition as described herein, wherein the food has a reduced glycemic index when compared to a reference food which is equivalent to the food except that the reference food does not comprise the composition. Optionally, the glycemic index of the food as compared to the reference food is reduced by at least 5, 10, 15, 20, 25, 30, 40 or 50% of the glycemic index of the reference food. Alternatively or additionally, the glycemic index of the food as compared to the reference food can be reduced by no more than 10, 15, 20, 25, 30, 40, 50 or 60% of the glycemic index of the reference food. It is also contemplated that the glycemic index of a food could be reduced to a level recognized in the art as medium or low, for example, a glycemic index of no more than 69 or no more than 55, respectively.

For example, starch and proteins in a fermented plant-origin material can interact in the presence of organic acid produced by microorganisms (e.g., yeast, bacteria, lactic-acid-producing microorganisms, or a combination thereof). After the interaction of the starch and proteins, the resultant starch and protein is less susceptible to fast amylase hydrolysis when compared to the starch alone before interaction with the protein. As a result, it is possible to reduce the glucose release rate during digestion and the glycemic index of a composition comprising fermented plant-origin material.

In some embodiments, the composition could enhance immunity of an individual after consumption.

In some embodiments, the composition provides sustained energy. For example, by slowing the rate at which glucose is released during digestion, the release of glucose can occur at a more consistent rate and contribute to a more sustained feeling of energy or lack of tiredness. In some embodiments of a composition as described herein, consumption of the composition by a human provides the human with a source of sustained energy.

For example, in some embodiments of a composition, available starch and protein in the composition have interacted under the influence of acid released during fermentation (e.g., lactic acid or other acid released by fermentation cultures or microorganisms). As an example, in some embodiments, upon heating fermented plant-origin material under elevated temperature conditions (e.g., those used in pasteurization, and/or 60-120° C.) lactic acid released by the fermentation cultures induces the interaction between available starch and protein. As a result of the interaction of the available starch and protein, a reduction occurs in the rate of the activity of amylase on the available starch in the composition.

For some embodiments, it is contemplated that the rate of reaction of amylase-catalyzed hydrolysis of the starch could be reduced by at least 5, 10, 15, 20, 30, 40, 50, 60, 70, 80, 90, 95, 96, 97, 98, 98.3, 99.4, or 99.5% relative to the rate of reaction of amylase-catalyzed hydrolysis of the starch. For example, without wishing to be bound by theory, it is contemplated that an amylase enzyme would typically catalyze a reference number of starch hydrolysis reactions in the reference food under a reference set of conditions (e.g., temperature, pressure, etc.) in vitro after consumption by a human. However, given a food which consists of a mixture of the reference food and a composition (e.g., as described herein and in which the available starch and protein have interacted in the presence of acid released during fermentation), it is contemplated that the same amylase enzyme under the same reference set of conditions in vitro in the human (except for those conditions related to the addition of the composition to the reference food), would catalyze a reduced number of starch hydrolysis reactions in the food.

In some embodiments, the reduced number of starch hydrolysis reactions is contemplated to be equal to at least 5, 10, 15, 20, 30, 40, 50, 60, 70, 80, 90, 95, 96, 97, 98, 98.3, 99, 99.1, 99.2, 99.3, 99.4, 99.5 or 99.75% less than the reference number of starch hydrolysis reactions. Accordingly, in some embodiments, it is contemplated that the rate of reaction of amylase-catalyzed hydrolysis of the starch in the food comprising the composition would be no more than (and/or no less than) 0.2, 0.25, 0.3, 0.4, 0.5, 0.6, 0.7, 0.8, 0.9 1, 1.7, 2, 3, 4, 5, 10, 20, 30, 40, 50, 60, 70, 80, 85, 90, or 95% of the rate of reaction of amylase-catalyzed hydrolysis of the starch in the reference food that does not comprise the composition.

As a skilled person would understand after reading this disclosure, although it can be desirable to reduce the rate of starch hydrolysis (e.g., enzyme catalyzed starch hydrolysis), it can be desirable to avoid slowing down the rate of starch hydrolysis too much. In some embodiments, it can be desirable for the food to be metabolized in a desired amount of time (e.g., 0.5 to 6, 1 to 5, 2 to 4, 3 hours or a range whose endpoints are selected from these values, and depending on whether the food is intended to be consumed for breakfast, for lunch, for dinner, as a snack, as a supplement, and the time expected until the next meal).

As a further illustration, if hydrolyzing a certain number of starch molecules only takes 1 minutes in a reference food, it is contemplated that hydrolyzing the same number of starch molecules could take 3 hours (i.e., 180 minutes) in the food comprising the composition if the rate of reaction of the amylase-catalyzed hydrolysis of the starch in the food composition were 0.56% (i.e., approximately 1/180) of the rate in the reference food.

In some embodiments, the composition comprises prebiotic microorganism, compounds, or a combination thereof. In some embodiments, the composition comprises live culture and/or microorganisms (e.g., live probiotic microorganisms), probiotic compounds, or a combination thereof. For example, the live microorganisms (e.g., live probiotic microorganisms) can be any component used as a fermenting agent 0117. The live microorganisms (e.g., live probiotic microorganisms) can also comprise additional probiotic microorganisms, for example, Lactobacillus plantarum LP299, Lactobacillus rhamosus LGG, Bifidobacterium animalis subsp. Lactis BB12, or a combination thereof. In some embodiments, the additional probiotic microorganisms can be added to the composition to support a sustained probiotic claim. In some embodiments, the additional probiotic microorganisms can be added to the composition after the fermentation step, for example, for the purpose (e.g., primary or exclusive purpose) of supporting a sustained probiotic claim. Examples of prebiotic and/or probiotic compounds include prebiotic and/or probiotic forms of beta-glucan.

In some embodiments, the composition can increase the soluble fiber per serving of a food, whether solid, liquid or semi-solid. For example, in some embodiments, the composition can comprise at least 0.75 g soluble beta-glucan fiber per serving, although the amount of soluble beta-glucan fiber per serving can also be an amount sufficient to make a heart health related claim according to relevant governmental, regulatory or certifying agencies. In some embodiments, the composition can comprise at least about 1.0 g soluble beta-glucan fiber per serving. As a skilled person would understand, serving size can be indicated by a product label for the composition, a customary serving size for the composition, or 240 mL of the composition in the absence of a specified serving size.

In some embodiments, the composition is a fiber source, soluble fiber source, nutrient additive, texture modifier, viscosity modifier or a combination thereof.

### Additional Embodiments

The following clauses are offered as further description of the disclosed invention:

1. A method comprising:

hydrolyzing a plant-origin material (e.g., in a hydrolysis reactor) to provide a hydrolyzed plant-origin material;

providing a fermentation starter material comprising the hydrolyzed plant-origin material (e.g., in a fermentation starter material mixer);

fermenting the fermentation starter material (e.g., in a fermentation reactor) to provide a fermented plant-origin material.

2. The method of any preceding clause, wherein the hydrolyzing comprises hydrolyzing starch in the plant-origin material.

3. The method of any preceding clause, wherein the hydrolyzing comprises hydrolyzing fiber in the plant-origin material.

4. The method of any preceding clause, wherein the hydrolyzing comprises hydrolyzing macronutrients selected from the group consisting of: starch, fiber, protein, and a combination thereof.

5. The method of any preceding clause,

wherein the hydrolyzing comprises hydrolyzing only a set of at least one macronutrient selected from the group consisting of: starch, fiber, protein, and a combination thereof; or

wherein the hydrolyzing comprises catalyzing hydrolysis with an enzyme that selectively hydrolyzes starch.

6. The method of any preceding clause, wherein the hydrolyzing does not comprise hydrolyzing a set of at least one macronutrient selected from the group consisting of: starch, fiber, protein, and a combination thereof.

7. The method of any preceding clause, wherein the plant-origin material is selected from the group consisting of: a grain, a cereal grain, a legume, a pulse, a pomace, a vegetable, a fruit, a plurality thereof (e.g., a plurality of types of grain, a plurality of types of legumes, etc.), and a combination thereof.

8. The method of any preceding clause, wherein the plant-origin material is selected from the group consisting of: a grain, a cereal grain, a legume, a pulse, a plurality thereof, and a combination thereof.

9. The method of any preceding clause, wherein the plant-origin material is selected from the group consisting of: a pomace, a vegetable, a fruit, a plurality thereof, and a combination thereof.

10. The method of any preceding clause, wherein the plant-origin material comprises protein, starch, fat, sugar, and beta-glucan.

11. The method of any preceding clause, wherein the plant-origin material comprises:

about 5 to about 40 wt. % protein;

about 0 to about 75 wt. % starch or about 0 to about 40 wt. % starch;

about 3 to about 30 wt. % total dietary fiber;

about 0 to about 7 wt. % sugar;

about 3 to about 15 wt. % fat; and

about 0 to about 20 wt. % beta-glucan.

12. The method of any preceding clause, comprising:

adding ingredients to the fermented plant-origin material (e.g., in an additional ingredient mixer), for example, to form a food product (e.g., solid food, liquid food, semi-solid/semi-liquid food, spoonable product, food bar, yogurt, soup, beverage, etc.).

13. The method of any preceding clause, comprising:

adjusting a moisture concentration of the fermented plant-origin material (e.g., in a moisture adjuster, for example, a mixer for adding water or a dryer for removing water) to provide a moisture-adjusted fermented plant-origin material (e.g., the moisture-adjusted fermented plant-origin material can be a food product, a powder, or a concentrate, for example, that can later be diluted to provide a beverage).

14. The method of any preceding clause, comprising:

drying the fermented plant-origin material (e.g., in a dryer that removes water from the fermented plant-origin material) to form a powder.

15. The method of any preceding clause, wherein the hydrolyzing comprises using an enzyme to catalyze the hydrolysis of starch in the plant-origin material.

16. The method of any preceding clause, wherein the hydrolyzing comprises using alpha-amylase to catalyze the hydrolysis of starch in the plant-origin material.

17. The method of any preceding clause, wherein the hydrolyzing comprises combining an enzyme with water and the plant-origin material to form a hydrolysis starting material, wherein the enzyme is used to catalyze hydrolysis of starch in the plant-origin material so that, after hydrolysis of the starch in the hydrolysis starting material to provide a hydrolyzed composition, the hydrolyzed composition comprises the hydrolyzed plant-origin material.

18. The method of clause 17, wherein the hydrolysis starting material comprises a total water mass concentration equal to about 25 to about 40 wt. %.

19. The method of clause 17, wherein the combining step lasts for about 1 to about 5 minutes or about 3 to about 5 minutes.

20. The method of clause 17, wherein the hydrolyzing comprises heating the hydrolysis starting material to a temperature equal to about 48 to about 100° C., or about 60 to about 83° C. to facilitate hydrolysis of the starch in the plant-origin material.

21. The method of any preceding clause wherein the hydrolyzing lasts for a time that reduces the average molecular weight of starch in the plant-origin material to a hydrolyzed starch average molecular weight that is about 0.07 to about 75% of the average molecular weight of the starch in the plant-origin material.

22. The method of any preceding clause wherein the hydrolyzing lasts for a time that reduces the peak molecular weight of starch in the plant-origin material to a hydrolyzed starch peak molecular weight that is about 6 to about 95% of the peak molecular weight of the starch in the plant-origin material.

23. The method of any preceding clause wherein the hydrolyzing lasts for about 0.5 to about 1.5 minutes or about 1 to about 1.5 minutes.

24. The method of clause 14 wherein the method comprises deactivating the alpha-amylase.

25. The method of clause 17, wherein the hydrolyzing step comprises deactivating the enzyme to provide the hydrolyzed plant-origin material (i.e., plant-origin material comprising starch that has been hydrolyzed under controlled conditions to reduce the molecular weight of the starch while substantially avoiding hydrolysis of the starch to non-starch components to within a specified tolerance).

26. The method of clause 25, wherein the deactivating step comprises heating the enzyme to a temperature sufficient to deactivate the enzyme, thereby providing the hydrolyzed plant-origin material.

27. The method of clause 25, wherein the deactivating comprises heating the enzyme to about 100 to about 180° C., or about 100 to about 130° C., thereby providing the hydrolyzed plant-origin material.

28. The method of clause 26, wherein the hydrolyzing the plant-origin material, the deactivating the enzyme, or a combination thereof comprises extruding the plant-origin material, the enzyme and optionally water in an extruder.

29. The method of clause 28, wherein the extruder is a twin-screw extruder.

30. The method of clause 28, wherein the extruder comprises a barrel with at least one heated barrel section, wherein a wall of at least one heated barrel section comprises a wall temperature equal to about 60 to about 166, about 137 to about 166, about 137 to about 152 or about 137 to about 150° C.

31. The method of clause 28, wherein the extruder comprises a barrel with a plurality of barrel sections, wherein each of the plurality of barrel sections comprises a wall temperature that differs from the wall temperature of the other barrel sections in the plurality of barrel sections.

32. The method of any preceding clause, comprising extruding the hydrolyzed composition through a die assembly of an extruder and optionally providing the hydrolyzed composition to the die assembly at a die pressure equal to about 1700 to about 11700 kPa and optionally at a die temperature equal to about 60 to about 166, about 137 to about 166 or about 140 to about 166° C., to form a hydrolyzed extrudate.

33. The method of clause 32, comprising pelletizing the hydrolyzed extrudate into pellets.

34. The method of clause 33, comprising milling the pellets to provide flour, optionally comprising drying the pellets to provide dried pellets before milling the dried pellets into the flour, optionally wherein the volume mean of the particles is 59 microns+/−50%.

35. The method of clause 32 or its dependent clauses, wherein the hydrolyzed extrudate comprises:

about 5 to about 40 wt. % protein;

about 0 to about 40 wt. % starch;

about 3 to about 30 wt. % total dietary fiber;

about 0 to about 7 wt. % of a combination of lactic acid and sugar;

about 3 to about 15 wt. % fat; and

about 0 to about 20 wt. % beta-glucan.

36. The method of any preceding clause wherein a mass ratio of starch:protein in the fermented plant origin material is equal to:

a mass ratio of starch:protein in the plant-origin material to within a tolerance of +/−30, 25, 20, 15, 10, 9, 8, 7, 6, 5, 4, 3, 2 or 1% of the mass ratio of starch:protein in the plant-origin material;

a mass ratio of starch:protein in the hydrolyzed plant-origin material to within a tolerance of +/−30, 25, 20, 15, 10, 9, 8, 7, 6, 5, 4, 3, 2 or 1% of the mass ratio of starch:protein in the hydrolyzed plant-origin material; or

a combination thereof.

37. The method of any preceding clause wherein a mass ratio of fat:protein in the fermented plant origin material is equal to:

a mass ratio of fat:protein in the plant-origin material to within a tolerance of +/−30, 25, 20, 15, 10, 9, 8, 7, 6, 5, 4, 3, 2 or 1% of the mass ratio of fat:protein in the plant-origin material;

a mass ratio of fat:protein in the hydrolyzed plant-origin material to within a tolerance of +/−30, 25, 20, 15, 10, 9, 8, 7, 6, 5, 4, 3, 2 or 1% of the mass ratio of fat:protein in the hydrolyzed plant-origin material; or

a combination thereof.

38. The method of any preceding clause wherein a mass ratio of beta-glucan:protein in the fermented plant origin material is equal to:

a mass ratio of beta-glucan:protein in the plant-origin material to within a tolerance of +/−30, 25, 20, 15, 10, 9, 8, 7, 6, 5, 4, 3, 2 or 1% of the mass ratio of beta-glucan:protein in the plant-origin material;

a mass ratio of beta-glucan:protein in the hydrolyzed plant-origin material to within a tolerance of +/−30, 25, 20, 15, 10, 9, 8, 7, 6, 5, 4, 3, 2 or 1% of the mass ratio of beta-glucan:protein in the hydrolyzed plant-origin material; or

a combination thereof.

39. The method of any preceding clause wherein at least about 30, 50, 60, 70, 80 or 90 wt. % of sucrose in the fermentation starter material is converted to lactic acid in the fermented plant origin material.

40. The method of any preceding clause wherein lactic acid makes up about 0 to 7 wt. % (and optionally at least 1, 2, 3, 4, 5 or 6 wt. %) of the fermented plant-origin material.

41. The method of any preceding clause wherein sufficient lactic acid is produced from the fermentation starter material to provide the fermented plant origin material with a pH of no more than 4.0, 3.9 or 3.8.

42. The method of clause 15 wherein the method comprises deactivating the enzyme (e.g., alpha-amylase) used to catalyze hydrolysis of the plant-origin material so that no more than 5, 4, 3, 2, 1, 0.9, 0.8, 0.7, 0.6, 0.5, 0.4, 0.3, 0.2, 0.1 or 0.0 wt. % of the starch in the plant-origin material has been converted to sugar in the hydrolyzed plant-origin material.

43. The method of any preceding clause, wherein the hydrolyzing comprises using at least one enzyme to catalyze the hydrolysis of at least one macronutrient in the plant-origin material, wherein the at least one macronutrient is selected from the group consisting of: starch, fiber, protein, and a combination thereof.

44. The method of any preceding clause, wherein the hydrolyzing comprises using at least one enzyme selected from the group consisting of: alpha-amylase, pectinase, cellulase, and a combination thereof.

45. The method of any preceding clause, wherein the method comprises deactivating the at least one enzyme so that no more than 5, 4, 3, 2, 1, 0.9, 0.8, 0.7, 0.6, 0.5, 0.4, 0.3, 0.2, 0.1 or 0.0 wt. % of the at least one macronutrient in the hydrolyzed plant-origin material has been converted to a component that no longer qualifies as the respective at least one macronutrient (e.g., starch or fiber can be converted to sugar and thus no longer qualify as starch or fiber).

46. The method of any preceding clause, wherein beta-glucan in the fermented plant-origin material is structurally unchanged from the structure of the beta-glucan in the plant-origin material before hydrolyzing the plant-origin material.

47. The method of any preceding clause, wherein beta-glucan in the fermented plant-origin material is structurally unchanged from the structure of the beta-glucan in the plant-origin material before fermenting the hydrolyzed plant-origin material.

48. The method of any preceding clause, wherein a mass proportion of beta-glucan in the fermented plant-origin material is not reduced relative to a mass proportion of beta-glucan in the intact plant-origin material from which the hydrolyzed plant-origin material is derived, wherein the mass proportion of beta-glucan in the fermented plant-origin material is calculated excluding any materials that have been added to the plant-origin material.

49. The method of any preceding clause, wherein the providing a fermentation starter material comprises:

adding an additional component to the hydrolyzed plant-origin material to provide the fermentation starter material, wherein the additional component is selected from the group consisting of: additional carbohydrates, additional proteins, additional lipids, additional vitamins, and additional minerals.

50. The method of any preceding clause, wherein the method comprises:

adding an additional plant-origin material to the hydrolyzed plant-origin material before the hydrolyzed plant-origin material is fermented, thereby providing the fermentation starter material, wherein the additional plant-origin material is selected from the group consisting of: a grain, a cereal grain, a pulse, a legume, a pomace, a vegetable, a fruit, a plurality thereof, and a combination thereof.

51. The method of any preceding clause, wherein the providing a fermentation starter material comprises:

adding an additional plant-origin material to the hydrolyzed plant-origin material, thereby providing the fermentation starter material, wherein the additional plant-origin material is selected from the group consisting of: a grain, a cereal grain, a legume, a pulse, a plurality thereof, and a combination thereof.

52. The method of any preceding clause, wherein the providing a fermentation starter material comprises:

adding an additional plant-origin material to the hydrolyzed plant-origin material before the hydrolyzed plant-origin material is fermented, thereby providing the fermentation starter material, wherein the additional plant-origin material is selected from the group consisting of: a pomace, a vegetable, a fruit, a plurality thereof, and a combination thereof.

53. The method of any preceding clause, wherein the additional plant-origin material is unhydrolyzed (e.g., has not been subject to intentional hydrolysis, has not been subject to significant hydrolysis, no more than 0.1, 0.2, 0.3, 0.4, 0.5, 1, 2, 3, 4, or 5 wt. % of at least one macronutrient (e.g., starch, protein, fiber, which can include cellulose and/or pectin, or a combination thereof) in the additional plant-origin material has been hydrolyzed, the average molecular weight of the at least one macronutrient (e.g. starch, protein, fiber, which can include cellulose and/or pectin, or a combination thereof) has decreased due to hydrolysis by no more than 0.1, 0.2, 0.3, 0.4, 0.5, 1, 2, 3, 4, or 5 wt. %, or a combination thereof).

54. The method of any preceding clause, wherein the additional plant-origin material is hydrolyzed (e.g., has been subject to intentional hydrolysis, has been subject to significant hydrolysis, at least 0.1, 0.2, 0.3, 0.4, 0.5, 1, 2, 3, 4, 5, 10, 20, 30, 40, 50, 60, 70, 80, 90, 95, 96, 97, 98, 99 or 100 wt. % of at least one macronutrient (e.g., starch) in the additional plant-origin material has been hydrolyzed, the average molecular weight of the at least one macronutrient (e.g., the starch) has decreased due to hydrolysis by at least 0.1, 0.2, 0.3, 0.4, 0.5, 1, 2, 3, 4, 5, 10, 20, 30, 40, 50, 60, 70, 80, 90, 95, 96, 97, 98, 99 wt. %, or a combination thereof).

55. The method of any preceding clause, wherein the method comprises:

adding a fermenting agent to the fermentation starter material to cause the fermenting of the fermentation starter material (e.g., in a fermentation slurry comprising the fermenting agent and the fermentation starter material)); wherein the fermenting agent is selected from the group consisting of yeast (e.g., Saccharomyces, Candida, Kluyveromyces), bacteria (e.g., Lactobacillus species, for example, Lactobacillus acidophilus, Lactobacillus delbruckii subsp. bulgaricus, Lactobacillus paracasei, Lactobacillus plantarum, Lactobacillus sanfrancisco, other lactic acid bacteria, for example, Streptococcus thermophiles, Bifidobacterium, Lactococcus species, Leuconostocs, Pediococcus, or a combination thereof), bacteria used for lactic acid fermentation, a bacteria that does not selectively hydrolyze beta-glucan, and a combination thereof.

56. The method of any preceding clause, wherein the fermenting occurs under fermentation conditions selected from the group consisting of: a pressure of 100-500, or 100-400, or 100-300, or 100-200, or 100-150 kPa (e.g. 101.325 kPa); temperature of 25-45, 25-40, 25-35, 25-30, 30-35, 35-40, 40-45, or 35-45° C.; stirring, mixing, or agitation; a pH of 5.5-7.8 at the start of fermentation; inoculating the fermentation starter material to provide an inoculated fermentation starter material (e.g., in a fermentation slurry comprising the inoculated fermentation starter material) comprising 10̂5-10̂8 colony forming units per milliliter (CFU/ml) of the inoculated fermentation starter material, wherein fermentation lasts for 1-36, 1-30, 1-25, 1-20, 1-15, 1-10, 1-5 hours or more than 36 hours.

57. The method of any preceding clause, wherein adding at least one ingredient to the fermented plant-origin material comprises:

adding an additional liquid to the fermented plant-origin material, wherein the additional liquid is selected from the group consisting of water, a dairy liquid, milk, a dairy milk, a non-dairy milk, a fruit-derived liquid material, a vegetable-derived liquid material, a vegetable juice, a fruit juice, a liquefied or pureed fruit, a liquefied or pureed vegetable, and a combination thereof.

58. The method of any preceding clause, wherein the plant-origin material comprises or is in the form of an extruded pellet or a flour (e.g., ground from an extruded pellet).

59. The method of any preceding clause, wherein water is added to the plant-origin material before the hydrolyzing the plant-origin material.

60. The method of any preceding clause, wherein the fermentation starter material comprises:

from about 5 to about 25 wt. %, 7 to 15 wt. % or about 10 to about 14 wt. % plant-origin material;

from about 0.5 to about 5 wt. % or about 1 to about 3 wt. % sucrose; and

from about 76 to about 96 wt. % added water.

61. The method of any preceding clause, wherein the fermenting occurs in a fermentation vessel.

62. The method of any preceding clause, wherein a fermentation slurry comprises the fermentation starter material and a fermenting agent (e.g., culture, yeast, bacteria or any combination thereof), wherein the fermentation slurry is fermented during the fermenting step to provide the fermented plant-origin material.

63. The method of any preceding clause, comprising mixing the fermentation starter material and fermentation culture at a mass ratio of about 5500:1 to about 4400:1, or optionally about 5000:1, to provide a fermentation slurry, wherein the fermentation slurry is fermented to provide the fermented plant-origin material.

64. The method of any preceding clause, wherein the fermentation slurry comprises about 0.018 to about 0.022 wt. %, or optionally about 0.020 wt. %, fermentation culture, and optionally wherein a fermentation slurry comprises about 99.982 wt. % to about 99.978, or optionally about 99.980 wt. %, fermentation starter material, wherein the fermentation culture comprises lactobacillus cultures.

65. The method of clause 33B, wherein the fermenting comprises agitating the fermentation slurry in a fermentation vessel, optionally wherein the agitating is caused by rotating a shaft having at least one protrusion, rotating a shaft having at least one paddle, rotating an auger, rotating an impeller, or a combination thereof at about 100 to about 400 rpm, 100 to about 200 rpm, or about 150 rpm in the fermentation slurry, optionally wherein the agitating lasts for about 10 to about 21 hours or about 15 to about 21 hours, optionally wherein the agitating occurs at about 35 to about 42° C. or about 40° C. and optionally wherein the agitating occurs at about atmospheric pressure.

66. The method of any preceding clause, wherein the fermented plant-origin material comprises a pH of no more than about 4.5, 4.2, 4.0, 3.9 or 3.8 and optionally no less than 2.0.

67. The method of any preceding clause, wherein the fermenting comprises:

adding yeast to the fermentation starter material (e.g., thereby providing a fermentation slurry comprising the fermentation starter material and the yeast) in a yeast fermentation step to provide the fermented plant-origin material with yeast-fermentation flavors.

68. The method of any preceding clause, wherein the fermenting comprises:

adding bacteria to the fermentation starter material (e.g., thereby providing a fermentation slurry comprising the fermentation starter material and the bacteria) in a bacterial fermentation step to provide the fermented plant-origin material with bacterial-fermentation flavors.

69. The method of any preceding clause, wherein the fermenting comprises a yeast fermentation step followed by a bacterial fermentation step; or wherein the fermenting comprises a yeast fermentation step and a bacterial fermentation step that occur simultaneously for at least 10%, 25%, 50%, 75%, 90%, 95% or all of the yeast fermentation step and/or bacterial fermentation step; or wherein the fermenting comprises a yeast fermentation step that starts before the bacterial fermentation step or that occurs simultaneously (e.g., coterminously or simultaneously to some extent) with the bacterial fermentation step.

70 The method of any preceding clause, wherein the adding at least one ingredient to the fermented plant-origin material comprises adding at least one ingredient selected from the group consisting of: a sweetener, sugar, sucrose, natural sweeteners, low calorie sweeteners, no calorie sweeteners, flavors (e.g, vanilla), a protein (e.g., plant protein or dairy protein), and a combination thereof.

71. The method of any preceding clause, wherein the method comprises:

heat-treating (e.g., pasteurizing) the fermented plant-origin material or the food product, for example, using a heat-treater, to provide a heat-treated product (e.g., shelf-stable product).

72. The method of any preceding clause, wherein the method comprises packaging and/or refrigerating the fermented plant-origin material, powder, or food product to provide a product comprising live culture and/or live microorganisms (e.g., live microorganisms with probiotic properties).

73. The method of any preceding clause, wherein the method comprises dehydrating (e.g., vacuum-dehydration, drying with heat, etc.) the fermented plant-origin material to provide a powder and optionally adding the powder to at least one food product ingredient (e.g., in a food product ingredient mixer) to provide a food product (e.g., solid food, liquid food, semi-solid/semi-liquid food, spoonable product, food bar, yogurt, soup, beverage, etc.), optionally wherein the powder comprises live culture and/or live microorganisms (e.g., live microorganisms having probiotic properties).

74. A composition formed by the method of any preceding clause.

75. A composition comprising:

a fermented plant-origin material (e.g., fermented, hydrolyzed plant-origin material provided by fermenting a hydrolyzed plant-origin material, and/or wherein the hydrolyzed plant origin material is provided by hydrolyzing a plant-origin material);

optionally, wherein the hydrolyzed plant-origin material comprises at least one hydrolyzed macronutrient selected from the group consisting of hydrolyzed starch, hydrolyzed fiber, hydrolyzed protein, and a combination thereof;

optionally, wherein the plant-origin material is selected from the group consisting of a grain, a cereal grain, a legume, a pulse, a pomace, a vegetable, a fruit, a plurality thereof, and a combination thereof.

76. A composition comprising:

fermented plant-origin material;

wherein the fermented plant-origin material comprises a fermentation product produced by fermenting fermentation starter material (e.g., in a fermentation slurry comprising the fermentation starter material), wherein the fermentation starter material comprises hydrolyzed plant-origin material.

77. The composition of any preceding clause, wherein the fermented plant-origin material comprises a pH of no more than 4.5, optionally no more than 4.0, 3.9 or 3.8 and optionally no less than 2.0.

78. The composition of any preceding clause, wherein the hydrolyzed plant-origin material comprises a Rapid Visco Analyzer (“RVA”) peak viscosity equal to no more than 2500 or 2000 cP and optionally at least 1, 5, 10, 15, 20, 25, 30, 40, 50, 60, 70, 80, 90, 100, 200, 300, 400, 500, 600, 700, 800, 900, 1000, or 1500 cP.

79. The composition of any preceding clause, wherein the fermented plant-origin material comprises a viscosity at 25° C. equal to no more than 7500 or 7000 cP and optionally at least 2000, 2500, 4500 or 5000 cP.

80. The composition of any preceding clause, wherein the fermented plant-origin material comprises a total water mass concentration equal to about 70 to 95 wt. %, about 70 to 90 wt. %, about 80 to 90 wt. %, or about 83.5 to about 86.5 wt. %.

81. The composition of any preceding clause, wherein the fermented plant origin material comprises a titratable acidity of about 0.3 to about 0.4 wt. %.

82. The composition of any preceding clause:

optionally, wherein the hydrolyzed plant-origin material comprises a hydrolysis product produced by hydrolyzing a plant-origin material,

optionally, wherein the hydrolyzed plant-origin material comprises a hydrolysis product produced by hydrolyzing at least one macronutrient in a plant-origin material, wherein the at least one macronutrient is selected from the group consisting of starch, fiber, protein, and a combination thereof; and

optionally, wherein the hydrolysis product comprises at least one hydrolyzed macronutrient selected from the group consisting of hydrolyzed starch, hydrolyzed fiber, hydrolyzed protein, and a combination thereof.

83. The composition of any preceding clause:

wherein the plant-origin material is selected from the group consisting of a grain, a cereal grain, a legume, a pulse, a pomace, a vegetable, a fruit, a plurality thereof, and a combination thereof.

84. The composition of any preceding clause,

wherein the hydrolyzed plant-origin material has been subject to intentional hydrolysis;

wherein the hydrolyzed plant-origin material has been subject to significant hydrolysis;

wherein at least 0.1, 0.2, 0.3, 0.4, 0.5, 1, 2, 3, 4, 5, 10, 20, 30, 40, 50, 60, 70, 80, 90, 95, 96, 97, 98, 99 or 100 wt. % of each of at least one macronutrient (e.g., starch) in the hydrolyzed plant-origin material has been hydrolyzed;

wherein the average molecular weight of each of the at least one macronutrient (e.g., the starch) in the hydrolyzed plant origin material has decreased due to hydrolysis by at least 0.1, 0.2, 0.3, 0.4, 0.5, 1, 2, 3, 4, 5, 10, 20, 30, 40, 50, 60, 70, 80, 90, 95, 96, 97, 98, 99 wt. %; or

a combination thereof.

85. The composition of any preceding clause, wherein the fermentation starter material (e.g., hydrolyzed plant-origin material) comprises:

an additional plant-origin material, wherein the additional plant-origin material is selected from the group consisting of: a grain, a cereal grain, a pulse, a legume, a pomace, a vegetable, a fruit, a plurality thereof, and a combination thereof.

86. The composition of any preceding clause, wherein the fermentation starter material comprises:

an additional plant-origin material, wherein the additional plant-origin material is selected from the group consisting of: a grain, a cereal grain, a legume, a pulse, a plurality thereof, and a combination thereof.

87. The composition of any preceding clause, wherein the fermentation starter material comprises:

an additional plant-origin material, wherein the additional plant-origin material is selected from the group consisting of: a pomace, a vegetable, a fruit, a plurality thereof, and a combination thereof.

88. The composition of any preceding clause, wherein the additional plant-origin material is unhydrolyzed, for example, has not been subject to intentional hydrolysis, has not been subject to a hydrolysis process, has not been subject to significant hydrolysis, no more than 0.1, 0.2, 0.3, 0.4, 0.5, 1, 2, 3, 4, or 5 wt. % of each of at least one macronutrient (e.g., starch) in the additional plant-origin material has been hydrolyzed, the average molecular weight of each of the at least one macronutrient (e.g., the starch) has decreased due to hydrolysis by no more than 0.1, 0.2, 0.3, 0.4, 0.5, 1, 2, 3, 4, or 5 wt. %, or a combination thereof.

89. The composition of any preceding clause, wherein the additional plant-origin material is hydrolyzed, for example, has been subject to intentional hydrolysis, has been subject to a hydrolysis process, has been subject to significant hydrolysis, at least 0.1, 0.2, 0.3, 0.4, 0.5, 1, 2, 3, 4, 5, 10, 20, 30, 40, 50, 60, 70, 80, 90, 95, 96, 97, 98, 99 or 100 wt. % of each of at least one macronutrient (e.g., starch) in the additional plant-origin material has been hydrolyzed, the average molecular weight of each of the at least one macronutrient (e.g., the starch) has decreased due to hydrolysis by at least 0.1, 0.2, 0.3, 0.4, 0.5, 1, 2, 3, 4, 5, 10, 20, 30, 40, 50, 60, 70, 80, 90, 95, 96, 97, 98, 99 wt. %, or a combination thereof.

90. The composition of any preceding clause, wherein the composition comprises at least one enzyme (e.g., deactivated enzyme) selected from the group consisting of: alpha-amylase, pectinase, cellulase and a combination thereof.

91. The composition of any preceding clause, wherein the fermentation starter material comprises at least one deactivated enzyme selected from the group consisting of: deactivated alpha-amylase, deactivated pectinase, deactivated cellulase and a combination thereof.

92. The composition of any preceding clause, wherein the composition comprises fermentation-derived molecules, optionally selected from the group consisting of: organic acids (e.g., lactic acid), esters, alcohols, aldehydes, ketones, antimicrobial molecules, and exopolysaccharides.

93. The composition of any preceding clause, wherein the plant-origin material is grain.

94. The composition of any preceding clause, wherein the plant-origin material and/or the hydrolyzed plant-origin material is whole grain.

95. The composition of any preceding clause, wherein the hydrolyzing comprises using beta-amylase and alpha-amylase to provide the hydrolyzed plant-origin material and wherein the hydrolyzed plant-origin material is not whole grain.

96. The composition of any preceding clause, wherein the hydrolyzed plant-origin material is derived from intact grain caryopses; wherein the average molecular weight of the hydrolyzed starch is reduced by at least 30%, 40%, 50%, 60% or 65% relative to the average molecular weight of the starch in the intact grain caryopses.

97. The composition of any preceding clause, wherein the hydrolyzed plant-origin material is derived from intact grain caryopses; wherein the intact grain caryopses comprise principal anatomical components; wherein the principal anatomical components comprise a starchy endosperm, a germ and a bran; wherein the principal anatomical components are present in a first set of relative component proportions in the intact grain caryopses; wherein the first set of relative component proportions comprises proportions selected from the group consisting of (i) the mass of starchy endosperm divided by the mass of germ, (ii) the mass of starchy endosperm divided by the mass of bran, (iii) the mass of bran divided by the mass of germ, (iv) the mass of any one principal anatomical component divided by the mass of any other principal anatomical component, and (v) a combination thereof; wherein the principal anatomical components are present in a second set of relative component proportions in the hydrolyzed plant-origin material; and wherein each proportion in the second set of relative component proportions in the hydrolyzed plant-origin material is equal to the corresponding proportion in the first set of relative component proportions in the intact grain caryopses +/−5, 4, 3, 2, 1, 0.9, 0.8, 0.7, 0.6, 0.5, 0.4, 0.3, 0.2, 0.1 or 0.0% of the corresponding proportion in the first set of relative component proportions.

98. The composition of any preceding clause, wherein the hydrolyzed plant-origin material comprises principal anatomical components comprising starchy endosperm, germ and bran; and wherein the principal anatomical components are present in the same, approximately the same, or +/−5, 4, 3, 2, 1, 0.9, 0.8, 0.7, 0.6, 0.5, 0.4, 0.3, 0.2, 0.1 or 0.0% relative proportions as they exist in the intact caryopses from which the hydrolyzed plant-origin material is derived.

99. The composition of any preceding clause, wherein the hydrolyzed plant-origin material is derived from intact grain caryopses; wherein the intact grain caryopses comprise principal nutrients; wherein the principal nutrients comprise starch, fat, protein, dietary fiber, beta-glucan, and sugar; wherein the principal nutrients are present in a first set of relative nutrient proportions in the intact grain caryopses; wherein the first set of relative nutrient proportions comprises proportions selected from the group consisting of (i) the mass of starch divided by the mass of fat, (ii) the mass of starch divided by the mass of protein, (iii) the mass of starch divided by the mass of dietary fiber, (iv) the mass of starch divided by the mass of beta-glucan, (v) the mass of starch divided by the mass of sugar, (vi) the mass of any one principal nutrient divided by the mass of another principal nutrient, and (vii) a combination thereof; wherein the principal nutrients are present in a second set of relative nutrient proportions in the hydrolyzed plant-origin material; and wherein each proportion in the second set of relative nutrient proportions in the hydrolyzed plant-origin material is equal to the corresponding proportion in the first set of relative proportions in the intact grain caryopses +/−5, 4, 3, 2, 1, 0.9, 0.8, 0.7, 0.6, 0.5, 0.4, 0.3, 0.2, 0.1 or 0.0% of the corresponding proportion in the first set of relative proportions.

100. The composition of any preceding clause, wherein the hydrolyzed plant origin material comprises principal nutrients comprising starch, fat, protein, dietary fiber, beta-glucan, and sugar; and wherein the principal nutrients are present in the same, approximately the same, or +/−5, 4, 3, 2, 1, 0.9, 0.8, 0.7, 0.6, 0.5, 0.4, 0.3, 0.2, 0.1 or 0.0% of the relative mass proportions as they exist in the intact caryopses from which the hydrolyzed plant-origin material is derived.

101. The composition of any preceding clause, wherein the composition and/or the hydrolyzed plant-origin material comprises 1 to 20, 1 to 15, 3 to 5, or 3.7 to 4 wt. % beta-glucan.

102. The composition of any preceding clause, wherein the hydrolyzed plant-origin material is derived from intact grain caryopses; wherein the intact grain caryopses comprise beta-glucan; and wherein the beta-glucan in the fermented plant origin material is structurally unchanged relative to the beta-glucan in the intact caryopses.

103. The composition of any preceding clause, wherein the plant-origin material is oat.

104. The composition of any preceding clause, wherein the plant-origin material is a flour.

105. The composition of any preceding clause, wherein the plant-origin material is a highly dispersible flour (e.g., highly dispersible in water so that there are no lumps of the flour in a mixture of the flour and water at 25° C. after stirring the mixture for 5 seconds).

106. The composition of any preceding clause, wherein the composition comprises at least about 0.75 g or at least about 1.0 g soluble beta-glucan fiber per serving (e.g., serving size as indicated by a product label for the composition, customary serving size, or 240 mL of the composition in the absence of a specified serving size).

107. The composition of any preceding clause, wherein at least 50, 60, 70, 80, 90, 95, 96, 97, 98, 99 or 100 wt. % of starch in the hydrolyzed plant-origin material is hydrolyzed starch.

108. The composition of any preceding clause, wherein the average molecular weight of the hydrolyzed starch in the hydrolyzed plant-origin material is 1.7-2.0×10⁶ Dalton.

109. The composition of any preceding clause, wherein no more than 5, 4, 3, 2, 1, 0.9, 0.8, 0.7, 0.6, 0.5, 0.4, 0.3, 0.2, 0.1 or 0.0 wt. % of starch in hydrolyzed plant-origin material has been converted to sugar.

110. The composition of any preceding clause, wherein the hydrolyzed plant-origin material is derived from intact plant-origin material; wherein the intact plant-origin material comprises principal nutrients; wherein the principal nutrients comprise starch, fat, protein, dietary fiber, beta-glucan, and sugar; wherein the principal nutrients are present in a first set of relative nutrient proportions in the intact plant-origin material; wherein the first set of relative nutrient proportions comprises proportions selected from the group consisting of (i) the mass of starch divided by the mass of fat, (ii) the mass of starch divided by the mass of protein, (iii) the mass of starch divided by the mass of dietary fiber, (iv) the mass of starch divided by the mass of beta-glucan, (v) the mass of starch divided by the mass of sugar, (vi) the mass of any one principal nutrient divided by the mass of another principal nutrient, and (vii) a combination thereof; wherein the principal nutrients are present in a second set of relative nutrient proportions in the hydrolyzed plant-origin material; and wherein each proportion in the second set of relative nutrient proportions in the hydrolyzed plant-origin material is equal to the corresponding proportion in the first set of relative mass proportions in the intact plant-origin material +/−5, 4, 3, 2, 1, 0.9, 0.8, 0.7, 0.6, 0.5, 0.4, 0.3, 0.2, 0.1 or 0.0% of the corresponding proportion in the first set of relative proportions.

111. The composition of any preceding clause, wherein the hydrolyzed plant-origin material comprises principal nutrients comprising starch, fat, protein, dietary fiber, beta-glucan, and sugar; and wherein the principal nutrients are present in approximately the same, the same or +/−5, 4, 3, 2, 1, 0.9, 0.8, 0.7, 0.6, 0.5, 0.4, 0.3, 0.2, 0.1 or 0.0% relative mass proportions as they exist in the intact plant-origin material 0102 (e.g., grain caryopses) from which the hydrolyzed plant-origin material is derived.

112. The composition of any preceding clause, wherein the composition is a beverage.

113. The composition of any preceding clause, wherein the composition comprises a mass concentration of fermented plant-origin material equal to 1-100%, 5-95%, 10-90%, 20-80%, 30-70%, 40-60%, 1-5%, 5-10%, 10-20%, 20-30%, 30-40%, 40-50%, 50-60%, 60-70%, 70-80%, 80-90%, 90-95%, 95-100%, or a combination thereof.

114. The composition of any preceding clause, wherein the composition comprises a mass concentration of hydrolyzed plant-origin material (e.g., grain, whole grain, legume or whole legume, pulse or whole pulse) equal to 1-100%, 5-95%, 10-90%, 20-80%, 30-70%, 40-60%, 1-5%, 5-10%, 10-20%, 20-30%, 30-40%, 40-50%, 50-60%, 60-70%, 70-80%, 80-90%, 90-95%, 95-100%, or a combination thereof.

115. The composition of any preceding clause, wherein the composition is a food product (e.g., flowable food product, liquid, beverage, semi-liquid, or combination thereof) and comprises a viscosity equal to 0.5 to 800, 0.5 to 700, 0.5 to 600, 0.5 to 500, 0.5 to 400, 0.5 to 300, 0.5 to 250, 0.5 to 200, 0.5 to 150, 0.5 to 100, 1 to 100, 0.5 to 50, 1 to 50, 0.5 to 30, or 1 to 30 cP at 25° C.

116. The composition of any preceding clause, wherein the composition comprises a liquid mass concentration equal to 0-99%, 5-95%, 10-90%, 20-80%, 30-70%, 40-60%, 0-5%, 5-10%, 10-20%, 20-30%, 30-40%, 40-50%, 50-60%, 60-70%, 70-80%, 80-90%, 90-95%, 95-99%, or a combination thereof.

117. The composition of any preceding clause, wherein the composition comprises a liquid selected from the group consisting of water, milk, a dairy milk, a non-dairy milk, a vegetable juice, a fruit juice, and a combination thereof.

118. The composition of any preceding clause, wherein the composition comprises an additional plant-origin material selected from the group consisting of a grain, a cereal grain, a pulse, a legume, a pomace, a vegetable, a fruit, a plurality thereof, and a combination thereof.

119. The composition of any preceding clause, wherein the composition comprises an additional ingredient selected from the group consisting of: additional carbohydrates, additional proteins, additional lipids, additional vitamins, additional minerals, and a combination thereof.

120. The composition of any preceding clause, wherein the composition is a prebiotic.

121. The composition of any preceding clause, wherein the composition is a glycemic index reducer that reduces the glycemic index of a food to which the glycemic index reducer is added by at least 5, 10, 15, 20, 25, 30, 40 or 50% or by no more than 10, 15, 20, 25, 30, 40, 50 or 60%, or a combination thereof; or

wherein the composition comprises a base food and a subcomposition comprising the fermented plant-origin material, wherein the subcomposition is a glycemic index reducer so that the glycemic index of the composition is reduced by at least 5, 10, 15, 20, 25, 30, 40 or 50 of the glycemic index of the base food or by no more than 10, 15, 20, 25, 30, 40, 50 or 60% of the glycemic index of the base food, or a combination thereof.

122. The composition of any preceding clause, optionally, wherein the composition could enhance immunity, optionally wherein the composition could have immunostimulatory effects (e.g., on a human, via the impact of lactic cultures and/or metabolites of lactic cultures produced during fermentation, for example, in the gastrointestinal ecosystem of the human), or a combination thereof.

123. The composition of any preceding clause, wherein consumption of the composition by a human provides the human with a source of sustained energy, wherein available starch and protein in the composition have interacted under the influence of acid released during fermentation (e.g., lactic acid) to reduce the rate of reaction of amylase-catalyzed hydrolysis of the starch.

124. The composition of any preceding clause, wherein the composition comprises live microorganisms (e.g., live microorganisms having probiotic properties), for example, selected from the group consisting of a component used as a fermenting agent, additional probiotic microorganisms (e.g., Lactobacillus plantarum LP299, Lactobacillus rhamosus LGG, Bifidobacterium animalis subsp. Lactis BB12, probiotic microorganisms added to support a sustained probiotic clause, or a combination thereof), and a combination thereof.

125. The composition of any preceding clause, wherein the composition is a fiber source (e.g., soluble fiber source).

126. The composition of any preceding clause, wherein the composition is a nutrient additive.

127. The composition of any preceding clause, wherein the composition is a texture modifier.

128. The composition of any preceding clause, wherein the composition is a viscosity modifier.

129. The composition of any preceding clause, wherein the composition comprises a live, dead, active, or inactive fermenting agent 0117 selected from the group consisting of yeast (e.g., Saccharomyces, Candida, Kluyveromyces), bacteria (e.g., Lactobacillus species, for example, Lactobacillus acidophilus, Lactobacillus delbruckii subsp. bulgaricus, Lactobacillus paracasei, Lactobacillus plantarum, Lactobacillus sanfrancisco, other lactic acid bacteria, for example, Streptococcus thermophiles, Bifidobacterium, Lactococcus species, Leuconostocs, Pediococcus, or a combination thereof), bacteria used for lactic acid fermentation, a bacteria that has no expressed beta-glucanase activity during fermentation, bacteria selected so that it expresses limited beta-glucan activity during fermentation so that the level of beta glucan in the composition after fermentation is at least (and/or no more than) 30, 40, 50, 60, 70, 80, 90, 95, 96, 97, 98, or 99 wt. % the beta-glucan present in the fermentation starter material that is fermented to provide the composition, and a combination thereof.

130. The composition of any preceding composition clause formed by the method of any preceding method clause.

131. A method or composition formed by combining one or more elements selected from any preceding clause or combination of clauses.

132. A food comprising the composition of any preceding clause, wherein the food has a reduced glycemic index when compared to a reference food, wherein the reference food is equivalent to the food except that the reference food does not comprise the composition, and optionally, wherein the glycemic index of the food as compared to the reference food is reduced by at least 5, 10, 15, 20, 25, 30, 40 or 50% of the glycemic index of the reference food or reduced to no more than 10, 15, 20, 25, 30, 40, 50 or 60% of the glycemic index of the reference food, or a combination thereof.

133. A food comprising the composition of any preceding clause:

optionally, wherein consumption of the food by a human provides the human with a more sustained source of energy relative to a reference food, wherein the reference food is equivalent to the food except that the reference food does not comprise the composition, wherein available starch and protein in the composition have interacted under the influence of acid released during fermentation (e.g., lactic acid) to reduce the rate of reaction of amylase-catalyzed hydrolysis of the starch;

optionally, wherein the rate of amylase-catalyzed hydrolysis of starch (e.g., available starch) in the food is no more than 0.2, 0.25, 0.3, 0.4, 0.5, 0.6, 0.7, 0.8, 0.9 1, 1.7, 2, 3, 4, 5, 10, 20, 30, 40, 50, 60, 70, 80, 85, 90, or 95% of the rate of reaction of amylase-catalyzed hydrolysis of the starch (e.g., available starch) in a reference food that does not comprise the composition, wherein the food is formed by combining the reference food and the composition, wherein the rate of reaction of amylase-catalyzed hydrolysis of the starch in the reference food occurs under a set of reference conditions (e.g., in vitro, in a human, body/specified temperature, body/specified pressure, or combination thereof) and wherein the rate of reaction of amylase-catalyzed hydrolysis of the starch in the food occurs under the set of reference conditions;

optionally, wherein the rate of amylase-catalyzed hydrolysis of starch (e.g., available starch) in the food is no less than 0.2, 0.25, 0.3, 0.4, 0.5, 0.6, 0.7, 0.8, 0.9 1, 1.7, 2, 3, 4, 5, 10, 20, 30, 40, 50, 60, 70, 80, 85, 90, or 95% of the rate of reaction of amylase-catalyzed hydrolysis of the starch (e.g., available starch) in a reference food that does not comprise the composition, wherein the food is formed by combining the reference food and the composition, wherein the rate of reaction of amylase-catalyzed hydrolysis of the starch in the reference food occurs under a set of reference conditions (e.g., in vitro, in a human, body/specified temperature, body/specified pressure, or combination thereof) and wherein the rate of reaction of amylase-catalyzed hydrolysis of the starch in the food occurs under the set of reference conditions; or

a combination thereof.

Although embodiments of the invention have been described with reference to several elements, any element described in the embodiments described herein are exemplary and can be omitted, substituted, added, combined, or rearranged as applicable to form new embodiments. A skilled person, upon reading the present specification, would recognize that such additional embodiments are effectively disclosed herein. For example, where this disclosure describes characteristics, structure, size, shape, arrangement, or composition for an element or process for making or using an element or combination of elements, the characteristics, structure, size, shape, arrangement, or composition can also be incorporated into another element or combination of elements, or process for making or using another element or combination of elements described herein to provide additional embodiments. Furthermore, it should be understood that the method steps described herein are exemplary, and upon reading the present disclosure, a skilled person would understand that one or more method steps described herein can be combined, omitted, re-ordered, or substituted.

Additionally, where an embodiment is described herein as comprising some element or group of elements, additional embodiments can consist essentially of or consist of the element or group of elements. Also, although the open-ended term “comprises” is generally used herein, additional embodiments can be formed by substituting the terms “consisting essentially of” or “consisting of.”

Also, when a range for a particular variable is given for an embodiment, an additional embodiment can be created using a subrange or individual values that are contained within the range. Moreover, when a value, values, a range, or ranges for a particular variable are given for one or more embodiments, an additional embodiment can be created by forming a new range whose endpoints are selected from any expressly listed value, any value between expressly listed values, and any value contained in a listed range. For example, if the application were to disclose an embodiment in which a variable is equal to 1 and a second embodiment in which the variable is equal to 3-5, a third embodiment can be created in which the variable is equal to 1.31-4.23. Similarly, a fourth embodiment can be created in which the variable is equal to 1-5. Although all ranges may or may not have the same or similar functions, modified ranges as described herein are contemplated and may also be inventive for the same, similar, or different reasons when compared to the expressly listed values and ranges.

As used herein, examples of “substantially” include: “more so than not,” “mostly,” and “at least 30, 40, 50, 60, 70, 80, 90, 95, 96, 97, 98 or 99%” with respect to a referenced characteristic.

As used herein, examples of “about” and “approximately” include a specified value or characteristic, plus or minus 30, 20, 10, 5, 4, 3, 2, or 1% of the specified value or characteristic.

While this invention has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this invention includes all modifications and equivalents of the subject matter recited in the claims appended hereto as permitted by applicable law. Moreover, any combination of the elements described herein, in all possible variations thereof, is encompassed by the invention unless otherwise indicated herein or otherwise clearly contradicted by context.

We claim:

1. A method comprising:
hydrolyzing a plant-origin material to provide a hydrolyzed plant-origin material;
providing a fermentation starter material comprising the hydrolyzed plant-origin material;
adding a fermenting agent to the fermentation starter material, wherein the fermenting agent comprises bacteria used for lactic acid fermentation;
fermenting the fermentation starter material to provide a fermented plant-origin material comprising fermentation metabolites, wherein the fermentation metabolites comprise lactic acid, and wherein the fermented plant-origin material has a pH equal to no more than 4.5.

2. The method of claim 1, wherein the hydrolyzing comprises hydrolyzing starch in the plant-origin material.

3. The method of claim 1, wherein the hydrolyzing comprises hydrolyzing fiber in the plant-origin material.

4. The method of claim 1, wherein the hydrolyzing protein in the plant-origin material.

5. The method of claim 1, wherein the hydrolyzing comprises catalyzing hydrolysis with an enzyme that selectively hydrolyzes starch.

6. The method of claim 1, wherein the plant-origin material is a cereal grain.

7. The method of claim 1, wherein the plant-origin material is oats.

8. The method of claim 1, wherein the plant-origin material comprises protein, starch, fat, sugar, and beta-glucan.

9. The method of claim 1, wherein the plant-origin material comprises:
about 5 to about 40 wt. % protein;
about 0 to about 40 wt. % starch;
about 3 to about 30 wt. % total dietary fiber;
about 0 to about 7 wt. % sugar;
about 3 to about 15 wt. % fat; and
about 0 to about 20 wt. % beta-glucan.

10. The method of claim 1, comprising:
adding ingredients to the fermented plant-origin material to form a food product.

11. The method of claim 1, comprising:
adjusting a moisture concentration of the fermented plant-origin material to provide a concentrate that can later be diluted to provide a beverage.

12. The method of claim 1, comprising:
drying the fermented plant-origin material to form a powder.

13. The method of claim 1, wherein the hydrolyzing comprises using an enzyme to catalyze the hydrolysis of starch in the plant-origin material.

14. The method of claim 1, wherein the hydrolyzing comprises using alpha-amylase to catalyze the hydrolysis of starch in the plant-origin material.

15. The method of claim 1, wherein the hydrolyzing comprises combining an enzyme with water and the plant-origin material to form a hydrolysis starting material, wherein the enzyme is used to catalyze hydrolysis of starch in the plant-origin material so that, after hydrolysis of the starch in the hydrolysis starting material to provide a hydrolyzed composition, the hydrolyzed composition comprises the hydrolyzed plant-origin material.

16. The method of claim 15, wherein the hydrolysis starting material comprises a total water mass concentration equal to about 25 to about 40 wt. %.

17. The method of claim 15, wherein the combining step lasts for about 1 to about 5 minutes.

18. The method of claim 15, wherein the hydrolyzing comprises heating the hydrolysis starting material to a temperature equal to about 48 to about 100° C. to facilitate hydrolysis of the starch in the plant-origin material.

19. The method of claim 1 wherein the hydrolyzing lasts for a time that reduces the peak molecular weight of starch in the plant-origin material to a hydrolyzed starch peak molecular weight that is about 6 to about 95% of the peak molecular weight of the starch in the plant-origin material.

20. The method of claim 1 wherein the hydrolyzing lasts for about 0.5 to about 1.5 minutes or about 1 to about 1.5 minutes.

21. The method of claim 14 wherein the method comprises deactivating the alpha-amylase.

22. The method of claim 1, comprising extruding the hydrolyzed composition through a die assembly of an extruder to form a hydrolyzed extrudate.

23. The method of claim 22, comprising pelletizing the hydrolyzed extrudate into pellets.

24. The method of claim 23, comprising milling the pellets to provide flour.

25. The method of claim 22, wherein the hydrolyzed extrudate comprises:
about 5 to about 40 wt. % protein;
about 0 to about 75 wt. % starch;
about 3 to about 30 wt. % total dietary fiber;
about 0 to about 7 wt. % of a combination of lactic acid and sugar;
about 3 to about 15 wt. % fat; and
about 0 to about 20 wt. % beta-glucan.

26. The method of claim 1 wherein a mass ratio of starch:protein in the fermented plant origin material is equal to:
a mass ratio of starch:protein in the plant-origin material to within a tolerance of +/−30% of the mass ratio of starch:protein in the plant-origin material.

27. The method of claim 1 wherein lactic acid makes up about 1 to 7 wt. % of the fermented plant-origin material.

28. The method of claim 1 wherein sufficient lactic acid is produced from the fermentation starter material to provide the fermented plant-origin material with a pH of no more than 4.0, 3.9 or 3.8.

29. The method of claim 15 wherein the method comprises deactivating the enzyme so that no more than 5 wt. % of the starch in the plant-origin material has been converted to sugar in the hydrolyzed-plant-origin material.

30. The method of claim 1, wherein the hydrolyzing comprises using alpha-amylase and cellulase.

31. The method of claim 1, wherein the hydrolyzing comprises using pectinase.

32. The method of claim 1, wherein beta-glucan in the fermented plant-origin material is structurally unchanged from the structure of the beta-glucan in the plant-origin material before hydrolyzing the plant-origin material.

33. The method of claim 1, wherein beta-glucan in the fermented plant-origin material is structurally unchanged from the structure of the beta-glucan in the plant-origin material before fermenting the hydrolyzed plant-origin material.

34. The method of claim 1, wherein a mass proportion of beta-glucan in the fermented plant-origin material is not reduced relative to a mass proportion of beta-glucan in the intact plant-origin material from which the hydrolyzed plant-origin material is derived, wherein the mass proportion of beta-glucan in the fermented plant-origin material is calculated excluding any materials that have been added to the plant-origin material.

35. The method of claim 1, wherein the providing a fermentation starter material comprises:
adding an additional component to the hydrolyzed plant-origin material to provide the fermentation starter material, wherein the additional component is selected from the group consisting of: additional carbohydrates, additional proteins, additional lipids, additional vitamins, and additional minerals.

36. The method of claim 1, wherein the method comprises:
adding an additional plant-origin material to the hydrolyzed plant-origin material before the hydrolyzed plant-origin material is fermented, thereby providing the fermentation starter material.

37. The method of claim 1, wherein the providing a fermentation starter material comprises:
adding an additional plant-origin material to the hydrolyzed plant-origin material, thereby providing the fermentation starter material, wherein the additional plant-origin material is a grain, a cereal grain, a legume, or a pulse.

38. The method of claim 1, wherein the method comprises:
wherein the fermenting agent comprises yeast.

39. The method of claim 1, wherein the fermenting occurs at a pressure of 100-500, kPa, a temperature of 25-45° C., at a starting pH of 5.5-7.8, while agitating a fermentation slurry comprising the fermentation starter material and the fermenting agent, and wherein the fermenting lasts for 1 to 36 hours.

40. The method of claim 1, wherein the method comprises:
adding an additional liquid to the fermented plant-origin material.

41. The method of claim 1, wherein the method comprises:
adding water to the plant-origin material before the hydrolyzing the plant-origin material.

42. The method of claim 1, wherein the fermentation starter material comprises:
from about 5 to about 25 wt. %, 7 to 15 wt. % or about 10 to about 14 wt. % plant-origin material;
from about 0.5 to about 5 wt. % or about 1 to about 3 wt. % sucrose; and
from about 76 to about 96 wt. % added water.

43. The method of claim 1, comprising mixing the fermentation starter material and fermentation culture at a mass ratio of about 5500:1 to about 4400:1 to provide a fermentation slurry, wherein the fermentation slurry is fermented to provide the fermented plant-origin material.

44. The method of claim 39, wherein the fermenting comprises agitating the fermentation slurry in a fermentation vessel, wherein the agitating is caused by rotating an impeller at about 100 to about 400 rpm in the fermentation slurry, wherein the agitating lasts for about 10 to about 21 hours, and wherein the agitating occurs at about 35 to about 42° C.

45. The method of claim 1, wherein the fermenting comprises a yeast fermentation step that starts before the bacterial fermentation step or that occurs simultaneously with the bacterial fermentation step.

46. The method of claim 1, wherein the hydrolyzed plant-origin material comprises a Rapid Visco Analyzer (“RVA”) peak viscosity equal to about 1 to 2500 cP.

47. A composition comprising:
fermented plant-origin material;
wherein the fermented plant-origin material comprises a fermentation product produced by fermenting fermentation starter material in a fermentation slurry comprising the fermentation starter material, wherein the fermentation starter material comprises hydrolyzed plant-origin material; and
wherein the fermented plant-origin material has a pH equal to no more than 4.5.

48. The composition of claim 47, wherein the fermented plant-origin material comprises a viscosity at 25° C. equal to no more than 7500 and at least 2000 cP.

49. The composition of claim 47, wherein the fermented plant-origin material comprises a total water mass concentration equal to about 80 to 90 wt. %.

50. The composition of claim 47, wherein the fermented plant origin material comprises a titratable acidity of about 0.3 to about 0.4 wt. %.

51. The composition of claim 47:
wherein the hydrolyzed plant-origin material comprises a hydrolysis product produced by hydrolyzing at least one macronutrient in a plant-origin material, wherein the at least one macronutrient comprises starch.

52. The composition of claim 47:
wherein the plant-origin material comprises a grain.

53. The composition of claim 47, wherein the fermentation starter material comprises:
an additional plant-origin material, wherein the additional plant-origin material comprises a pomace.

54. The composition of claim 47, wherein the additional plant-origin material is unhydrolyzed.

55. The composition of claim 47, wherein the composition comprises deactivated alpha-amylase.

56. The composition of claim 47, wherein the composition comprises fermentation metabolites comprising lactic acid.

57. The composition of claim 47, wherein the hydrolyzed plant-origin material is whole grain.

58. The composition of claim 47, wherein the hydrolyzed plant-origin material is derived from intact grain caryopses; wherein the average molecular weight of the hydrolyzed starch is reduced by at least 30% relative to the average molecular weight of the starch in the intact grain caryopses.

59. The composition of claim 47, wherein the hydrolyzed plant-origin material is derived from intact grain caryopses; wherein the intact grain caryopses comprise principal anatomical components; wherein the principal anatomical components comprise a starchy endosperm, a germ and a bran; wherein the principal anatomical components are present in a first set of relative component proportions in the intact grain caryopses; wherein the first set of relative component proportions comprises (i) the mass of starchy endosperm divided by the mass of germ, (ii) the mass of starchy endosperm divided by the mass of bran, (iii) the mass of bran divided by the mass of germ; wherein the principal anatomical components are present in a second set of relative component proportions in the hydrolyzed plant-origin material; and wherein each proportion in the second set of relative component proportions in the hydrolyzed plant-origin material is equal to the corresponding proportion in the first set of relative component proportions in the intact grain caryopses to within a tolerance of +/−5% of the corresponding proportion in the first set of relative component proportions.

60. The composition of claim 47, wherein the hydrolyzed plant-origin material is derived from intact grain caryopses; wherein the intact grain caryopses comprise principal nutrients; wherein the principal nutrients comprise starch, fat, protein, dietary fiber, beta-glucan, and sugar; wherein the principal nutrients are present in a first set of relative nutrient proportions in the intact grain caryopses; wherein the first set of relative nutrient proportions comprises (i) the mass of starch divided by the mass of fat, (ii) the mass of starch divided by the mass of protein, (iii) the mass of starch divided by the mass of dietary fiber, (iv) the mass of starch divided by the mass of beta-glucan, and (v) the mass of starch divided by the mass of sugar; wherein the principal nutrients are present in a second set of relative nutrient proportions in the hydrolyzed plant-origin material; and wherein each proportion in the second set of relative nutrient proportions in the hydrolyzed plant-origin material is equal to the corresponding proportion in the first set of relative proportions in the intact grain caryopses +/−5% of the corresponding proportion in the first set of relative proportions.

61. The composition of claim 47, wherein the composition comprises 1 to 20 wt. % beta-glucan.

62. The composition of claim 47, wherein the hydrolyzed plant-origin material is derived from intact grain caryopses; wherein the intact grain caryopses comprise beta-glucan; and wherein the beta-glucan in the fermented plant origin material is structurally unchanged relative to the beta-glucan in the intact caryopses.

63. The composition of claim 47, wherein the plant-origin material is whole grain oat flour.

64. The composition of claim 47, wherein the composition comprises at least 0.75 g, optionally at least 1.0 g, soluble beta-glucan fiber per serving.

65. The composition of claim 47, wherein at least 50 wt. % of starch in the hydrolyzed plant-origin material is hydrolyzed starch.

66. The composition of claim 47, wherein the average molecular weight of the hydrolyzed starch in the hydrolyzed plant-origin material is 1.7-2.0×10⁶ Dalton.

67. The composition of claim 47, wherein the composition is a beverage.

68. The composition of claim 47, wherein the composition comprises a mass concentration of fermented plant-origin material equal to 1-100%.

69. The composition of claim 47, wherein the composition comprises a mass concentration of hydrolyzed plant-origin material equal to 1-100%.

70. The composition of claim 47, wherein the composition is a food product and comprises a viscosity equal to 0.5 to 800 cP at 25° C.

71. The composition of claim 47, wherein the composition comprises a liquid mass concentration equal to 40-60%.

72. The composition of claim 47, wherein the composition comprises an additional plant-origin material comprising a pulse.

73. The composition of claim 47, wherein the composition comprises an additional comprising additional carbohydrates.

74. The composition of claim 47, wherein the composition is a prebiotic.

75. The composition of claim 47, wherein the composition comprises a base food and a subcomposition comprising the fermented plant-origin material, wherein the subcomposition is a glycemic index reducer so that the glycemic index of the composition is at least 5% less than the glycemic index of the base food.

76. The composition of claim 47, wherein consumption of the composition by a human provides the human with a source of sustained energy, wherein available starch and protein in the composition have interacted under the influence of acid released during fermentation to reduce the rate of reaction of amylase-catalyzed hydrolysis of the starch.

77. The composition of claim 47, wherein the composition comprises live microorganisms comprising probiotic microorganisms.

78. The composition of claim 47, wherein the composition comprises soluble fiber.

79. The composition of claim 47, wherein the composition is a nutrient additive.

80. The composition of claim 47, wherein the composition is a texture modifier.

81. The composition of claim 47, wherein the composition is a viscosity modifier.
